SEQUENCE LISTING (6 sequences)

SEQ ID NO: 1 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - DNA, 5093 bp, Homo sapiens
acgcacagcccccctgggggccgggggcggggccaggctataaaccgccggttaggggccgccatcccctcagagcgtcgggatatcgggtggcggctcgggacggaggacgcgctagtgttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaa

SEQ ID NO: 3 - protein, 418 aa, Homo sapiens
MESLTDPSKLDSGKEPHISLIPNKQDRTLTIVDTGIGMTKADLINNLGTITKSETKVFMEVLQAGADISMIGQFSVGFYSAYSVAEKVTVITKHNNDEQYAWESSLRGSFTEYREFYKSLTINWEDYLAVKHFSVEGQLEFRAFLFVPRLAPFELLETRKKKNKIKLSARRDLIMDNCEELIPEYLNFIRGVVDSEDLPLNIFRETKDQVANSTIVQRLWKHGLEVIYTIEPIDEYCVQQLKEFEGKTLVSVTKEDLELPEDEEEKKKQEEGKQKTKQKKNQSLRTSAKSTYGWTANMERIMKAQALRDNSTTGYMAAKKHLEINPDHSFIDTLRQKAETDKNDKSVKDLVILLYETALLSSDFGLEGPQTHANRIYRMNKLGLGTDEDDPTADDTSAAVTEEMPPLEGDDDTSRMEK

SEQ ID NO: 4 - DNA, 8210 bp, Homo sapiens
gagctccggctgccctgcactggttcccagagactccctccttcccaggtccaaatggctgcaggagcgaagtgggcggaaaaaaagcgaaccagcttgagaaagggcttgacgtgcctgcgtagggagggcgcatgtccccgtgctccgtgtacgtggcggccgcaggggctagaggggggtcccccccgcaggtactccactctcagtctgcaaaagtgtacgcccgcagagccgccccaggtgcctgggtgttgtgtgattgacgcggggaaggaggggtcagccgatccctccccaaccctccatcccatccctgaggattgggctggtacccgcgtctctcggacaggtcagagcgggtcgccgggtggggtcgctgcaaaaaccctgccccggccgcagccgagaggcggacgtcgcggggagggggcgggaccgccgagacaggcctggaaactgctggaaatgccgcagtgccgccgccgccccttccgccgcatgtcggcaaagagtccccgccagccccggccggcgccctccccctacgctgagctgcccctcagcgcgaaccctccgcccttcctctactcctgcgagagtcgggatctggggctacccaaggttgggtcccgaatgccagtccctctgtcgggacgcgagatgtgtagggcagatgctaggaagaagattgggtctgggacgggtggtccgcgtggttagctgcctccgctctttttcggtgtcccccccagtcccgcccttgggtgtggggacgcctgccccacaagtgtttagggaggtcagtgggttcctcgcccgtagagacaccgtttatgccaaatgagcactcctcatccccgctcttgatggagtcatgtcctagacgtgaaactatggggctgtgatcacaagcaaatgtgtgggcggatccgttgcttgggttcttccccgccccctctttttttcggaccatgacgtcaaggtgggctggtggcggcaggtgcggggttgacaatcatactcctttaaggcggagggatctacaggagggcggctgtactgtgcttcgccttatatagggcgacttggggcacgcagtagctctctcgagtcactccggcgcagtgttgggactgtctgggtatcggaaagcaagcctacgttgctcactattacgtataatccttttcttttcaaggtaaggctgagatctccgctaggcttctttccctttagtgctgtattcgtgttgtttttgtttttttctgtcctttagggagccttagtctagatgtcggggtggcttgtggataacgctctggatttttatagggtgagggtagtggtgggtgaggttttttgagtcctcctcggttttctctagtgtgtttggggggtggggctttctctcggcgcctgctggccgtagcgaggtgggctgtggggttggggcagtgggcggctggcagctgcacgtggtggccgcgcggcccgggacgctgccatttttgcccctccacttccggacgcggctacggggcgtcggagggggaccgcaggtggcgggggtgcccgctcgggtgactcagcacggccttgtgggactggctttgtcacctctcttatcggacgcgttgttaaagccttcttgggtgctttgtttctgtgagggagggttgacggtgtgggaagagagctttcggtctccagcacccgatactccctccttccagatctttcttgcagtcccggtggaggaggggcggggaggggagcaggttctggaagattcatgggctccttcctccgcccttcctcgagagctgagattgttctggaagcttctggattctggcgccccgccccagtgcccggatgctgggggcgagggagggtgcactgcggcgccccctcctcgcgtggtcctggccgacgcatgtccggcagtgacgagtgtcggcctggtggctacggccaccatctttcttgggtttggtcctgttctgtaattttgtgctgtgaaagggtcgtggtggagcttttggcttaagaattctttgtccggatttaattgctcctccggtgggtatcgtatggatcccaggttattcctccctgccctatgggcaggagtgtcccgcccttggactggtcttaggaactgacacctcagggggagcagtttaaagttagtgccatttttatcttaaactagtcactttgacctcccccaaataaagaactgtaggtagtgattttcacatttaaatttgtgtaaggattacttgggatctctagatacctgggttggaccaacattatgatttttctgccatactaccagatgatgctgaggctgctggtcaccattctttaagtaggtgggttctgtgacatttggttgaagaatatttagcttattttctttttccttctgaattttcaggcctcccacttagtgtgtagtctgagatctttaagagaatgcatttttagtcttgggaagggatagtactccggttaaaccagtctgaactcactgtctaaggtcctaacaaatgatatgacctttaggatttttaaacatggggccttagtgttcttttgtaattaatgagatttttattttagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgtaggtgctctggtttccacatttggcatggtttttttttttgatactctagaaggaggggaaaggagtggtttggcctttgttggggactactattgaagggggtaaacttgcagctattccaaaaagatgggttttactctggccatcttgaacttggaagggactatgtccagaataagtgggctcatggaactaactggttctaaagcctcaagatagggggcaatcagatttgaggctgagagaggtaaaccaagattttctttgaagatacgggctttaagaaagcaaaagtggctgagcgtgttggctcacacctgtaatgccaaggcaggaggatcacttgagcctaggatttcgagggcagcctgggcaaccgcgagaccttgtctctgcaaaaaattaaatattagttgggcacggtggcatgtgctgtagtcccagctacttgggaagctggggttgggaggatggctcgaacctgggaggtcaaggctgcagtgagctgtcatccttgccactgcactgtagcttgggcaacagagcaagagtctgtcttggaaagagcaaaagtaagttgctgtttgtatttccaggccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggtattgcagttctgtaggcattcatacttatctgtgttctttggttttttgcttctttaaaacttgtgattgactttaaacttgttggcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtaagttagcttttctgttacaaggtagttgggttaggattttctgggctcacaccagtagcagaaattttgggcatcctgtctgtaaagcagttcttcacagcagttctgctgatacttactaattgctggtctcaactgcatatactttttaccctgttacacgcttgtaattgactcttctaggtgagcccattggcaggggtaccaaagtgatcctccatcttaaagaagatcagacagagtacctagaagagaggcgggtcaaagaagtagtgaagaagcattctcagttcataggctatcccatcaccctttatgtgagtatggacttttaaatcttttacacttaacgtgcaggatgtttcctgttctggagaatctcattgtccctggcttttgctttccctggtagtgttttgtactccaaggctaacttctgtttttgttacttagttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaaggtgtgagaagcctttgcatgttggctcaacatgcacatatggagaggaatgagttaggtggaagagtgttgggtaatagacacacggaacttgtccaactgataacagaaatgtgatagccatgtgatttcacttactgattaccctgtcatagtgaagtgccatcatttctaatgacctcactttctcttcttatggaaatctgggtaatgtctattggcagccttacacatccagggttctgatcagaggggactgttttctacatacagctagtacccatctagatcgtggagggcattaaggctcagttttctcaggagctgcttgttgtgtgtgctctatcccttaggcttagggaggatcattgttccacttttagataatttggtgttggggctaaaaggtcctcttttgaaatgtaccacttatttttggtttctttcagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcagtgagtatctccttggcctaatttagttgggtgaagtcttgggaggttttaggcattctgctaggatattctaaggtaacagttttctgcaatacatagtaggtgtaagggttcaggaggctattagagccttctgtttgaatctggggaccaggtctggtctagctgtttttactgagctttctcaccctggttgatggcagattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaaggtaaaaaggcaaataatgcttattccctttaccactttcttagtaataacaataaattattccattcacattgaaagtgaagttattgtagttaagctggattgtttttcctcttcccacccttcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgcgttgactctgattgaagcctttttggaggagtggggagcacaattagggcttcctgggaactggcagtatgaggcattttagtcactgagttcatttaattaccctacaggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtaagccattctggggctaggatatattttgtaacatcttcgaggtgggctccctcacaagcatgtttctatacaattagtggtttgaggcagcctatttactgtttcatgccttcttgcctcttgttctcttctctagtcaggtttaaggctattttaataaaatttggcacagattaggcattgcttcagttaacttctgagagtagataaaataccatcattttcttttttttttctttttttgagatggggtctcgctctgtcacccaggctggagtgcagtggcacgatctctgctcattgcaagctccgcctcctgggttcacgccattctcctgccttagcctcctgagtagctgccactacaggcgcccgccaccacaccccggctaattttttgtatttttagtagagatggggtttcatcgcgttagccaggatggtctccatctcctgaccttgtgattcgcccacctcggcctcccaaagtgctgggattaacaggcgcaagccaccatgcctggccgattttttttttttttggactggatctcgctcactgcaaactcaagtctcctgagtggctgggattacagatgtgtgctaccacacccggttaattttttgtagacagggttttgccatgttggccagcatggtctcaaactcaagtggtctgtccacctcctccccctgctggaattaggcttgacaatgcctgttttctctttcaaagtggtaatgtcaatctaaggcttttgtgatcgtccacaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtaagtagctttggtacttggtgtggcaaggagtttgtgcaactcgtctcctctatggatttgacttaatgctatttggtcaagtctcacatggcttaattttacttcaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttatacagttctgctttcccttgtgaagtggatgttatccttccctagcttcttcatccctccagctcttgctgttttcatgagcacagcaagttgagctggttttgtagtgaaaataacagaataccagtgagtcttaagagttcacacactgaagctaaaggcagtttggaaaaactaccatataataatgccctttcagtcaaccaaaacacaggaccaagtccactgcagtaatttaatttaataaaataaaattataagagcaaaaagttacatttctaaagtaccaaaacctgcaacaggctcatggaacagagcctagggatcc

SEQ ID NO: 5 - protein, 290 aa, Homo sapiens
MVIQKEKKSCGQVVEEWKEFVWNPRTHQFMGRTGTSWAFILLFYLVFYGFPTAMFTLTMWVMLQTVSDHTPKYQDRLATPGLMIRPKTENLDVIVNVSDTESWDQHVQKLNKFLEPYNDSMQAQKNDVCRPGRYYEQPDNGVLNYPKLACQFNRTQLGNCSGIGDSTHYGYSTGQPCVFIKMNRVINFYAGANQSMNVTCAGKRDEDAENLGNFVMFPANGNIDLMYFPYYGKKFHVNYTQPLVAVKFLNVTPNVEVNVECRINAANIATDDERDKFAGRVAFKLRINKT

SEQ ID NO: 6 - DNA, 2208 bp, Homo sapiens
gaattcatgctaaattgctggaaggctgcgtctctgctgtggtgtcagttccggatgcctcatcgccaggggcgcgccgcagccacccaccctccggaccgcggcagctgctgacccgccatcgccatggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccaggattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcgatgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattgaagttaagagctgatcacaagcacaaatctttcccactagccatttaataagttaaaaaaagatacaaaaacaaaaacctactagtcttgaacaaactgtcatacgtatgggacctacacttaatctatatgctttacactagctttctgcatttaataggttagaatgtaaattaaagtgtagcaatagcaacaaaatatttattctactgtaaatgacaaaagaaaaagaaaaattgagccttgggacgtgcccatttttactgtaaattatgattccgtaactgaccttgtagtaagcagtgtttctggcccctaagtattgctgccttgtgtattttatttagtgtacagtactacaggtgcatactctggtcatttttcaagccatgttttattgtatctgttttctactttatgtgagcaaggtttgctgtccaaggtgtaaatattcaacgggaataaaactggcatggtaattttttttttttgtttgttttttgttttttggctctttcaaaggtaatggcccatcgatgagcatttttaacatactccatagtcttttcctgtggtgttaggtctttatttttatttttttcctgggggctggggtgggggtttgtcatgggggaactgccctttaaattttaagtgacactacagaaaaacacaaaaaggtgatgggttgtgttatgcttgtattgaatgctgtcttgacatctcttgccttgtcctccggtatgttctaaagctgtgtctgagatctggatctgcccatcactttggcctagggacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaagcaatgcagtaccccatagactggtgttaaatgttgtctacagtgcaaaatccatgttctaacatatgtaataattgccaggagtacagtgctcttgttgatcttgtattcagtcaggttaaaacaacggacaataaaagaatgaaccgaattc

(19) United States
(12) Patent Application Publication
(10) Pub. No.: US 2019/0375819 A1
(43) Pub. Date: Dec. 12, 2019

(54) GLYCOSYLATED TRANSFERRIN RECEPTOR 1 TUMOR ANTIGEN

(71) Applicant: Rhode Island Hospital, Providence, RI (US)

(72) Inventors: Jisu Li, Barrington, RI (US); Jason Shapiro, Pawtucket, RI (US); Jack R. Wands, East Greenwich, RI (US)

(21) Appl. No.: 16/346,061

(22) PCT Filed: Oct. 30, 2017
(86) PCT No.: PCT/US2017/059062

§ 371 Date: Apr. 29, 2019

Related U.S. Application Data
(60) Provisional application No. 62/414,662, filed on Oct. 29, 2016.
(60) Provisional application No. 62/415,204, filed on Oct. 31, 2016.

(51) Int. Cl. C07K 14/705; C07K 16/28; A61K 47/68; G01N 33/574; A61P 35/00; A61K 51/10; A61K 49/00

(52) U.S. Cl. CPC C07K 14/70582; C07K 16/2881; A61K 47/6849; G01N 33/57492; A61K 47/6859; C07K 2317/41; A61K 51/1027; A61K 51/1057; A61K 49/0058; C07K 2317/92; A61P 35/00

(57) ABSTRACT

This invention relates to compositions and methods for treating or diagnosing cancer.

### CROSS-REFERENCE TO RELATED APPLICATIONS

This application claims the benefit of priority to U.S. Provisional Application No. 62/414,662, filed Oct. 29, 2016, and 62/415,204 filed Oct. 31, 2016, which are hereby incorporated in their entireties and for all purposes.

### STATEMENT AS TO FEDERALLY SPONSORED RESEARCH

This invention was made with government support awarded by the National Institute of Health (NIH) under grant numbers T32DK066415, CA123544, and R21AI107618. The government has certain rights in the invention.

### FIELD OF THE INVENTION

This invention relates to treatment and/or diagnosis of cancer.

### BACKGROUND OF THE INVENTION

Hepatocellular carcinoma (HCC) is one of the most devastating cancers in the world. It is highly chemoresistant with no effective therapies. Early detection is crucial for timely treatment as well as for prevention of cancer progression and metastasis, so as to reduce morbidity and mortality. Currently, detection of pre-cancerous lesions or the early stage of HCC remains challenging. In view of the foregoing, there is an urgent need to develop reliable cancer biomarkers, which may serve as therapeutic targets as well.

### SUMMARY OF THE INVENTION

The invention addresses this need and features compositions, methods, and the use of purified human transferrin receptor 1 (TFR1), e.g., a purified cell surface glycosylated peptide, as a biomarker for a malignancy, for immunotherapy and/or as a vehicle for delivery of anti-tumor compounds, and/or a diagnostic agent. In some embodiments, the antigen, TFR1, is in the absence of other compounds with which it naturally occurs in the mammalian, e.g., human body, such as Heat Shock Protein 90 (HSP90) and/or Na+/K+ATPase or Mg++ ATPase (Transporting ATPase). To diagnose or prognose a malignancy, e.g., presence of a tumor, a TFR1 protein comprising an aberrant glycosylation is detected. For example, the glycosylation pattern differs from the glycosylation pattern of a normal wild type TFR1. The protein sequence where the abnormal glycosylation site resides is the result of the malignant transformation process. Such an aberrant glycosylation site is detected using an agent, e.g., an antibody such as a monoclonal antibody (optionally a humanized monoclonal antibody) that binds to the TFR1 protein at an epitope at or proximal to the aberrantly glycosylated site.

In one aspect, the composition or method comprises a purified human TFR1 protein or a purified cell surface glycosylated peptide fragment thereof, for use as a biomarker for detection of malignancy. For example, the protein or peptide fragment thereof comprises glycosylation at an aberrant site compared to a wild type TFR1 protein, e.g., the protein or peptide fragment does not comprise a N-linked glycosylation at amino acid position 251, 317, or 727 of SEQ ID NO:1. For example, the protein or peptide fragment comprises an N-linked glycosylation at amino acid position 50 and/or 55 of SEQ ID NO:1. In another example, the protein or peptide fragment comprises an 0-linked glycosylation at amino acid position 104 of SEQ ID NO:1.

Also within the invention is a purified monoclonal antibody that binds to a human transferrin receptor 1 for use in immunotherapy, as a vehicle for delivery of anti-tumor compounds, or as a diagnostic agent, wherein the antibody binds to an aberrant glycosylation site of the receptor. For example, the TFR1 comprises glycosylation at an aberrant site compared to a wild type TFR1 protein, e.g., as described above. In some embodiments, the antibody comprises AF-20; in other embodiments, antibody does not comprise AF-20.

In some cases, the TFR1 or glycosylated peptide thereof, is in the absence of other compounds with which it naturally occurs in a mammal, e.g., compounds being selected from the group consisting of Heat Shock Protein 90 (HSP90) and/or a Na+/K+ ATPase or Mg++ ATPase (Transporting ATPase). In other examples, the TFR1 exists in a complex with HSP90 and/or Transporting ATPase.

A method for delivering a cargo molecule to a subject, is carried out by administering an antibody conjugated to the cargo molecule to the subject, wherein the antibody binds to a glycosylated human transferrin-1 antigen, and wherein the cargo molecule is selected from the group consisting of a therapeutic agent or a detectable label. For example, the therapeutic agent comprises a cytotoxic compound. In another example, detectable label comprises a fluorescent compound or a radioisotope. The subject is characterized as comprising a malignancy (e.g., a tumor or other cell proliferative disorder), suspected of having a malignancy, or at risk of comprising a malignancy. For example, a subject suspected of having a malignancy or at risk of developing a malignancy may exhibit symptoms of a cancerous condition, may have a family history of a tumor type, or may be diagnosed as comprising a genetic sequence or epigenetic marker indicative of a cancerous state. In some examples, the method includes the use of AF-20 antibody; alternatively, the method does not comprise AF-20 antibody.

The invention encompasses a purified glycosylated TFR1 antigen epitope comprising N-linked glycosylation at amino acid position 50 of SEQ ID NO:1 or amino acid position 55 of SEQ ID NO:1 and the use of the epitope to make antibodies for cancer diagnostic and therapeutic use. The invention also encompasses a purified glycosylated TFR1 antigen epitope comprising O-linked glycosylation at amino acid position 104 of SEQ ID NO:1 and the use of such an epitope for generating antibodies for cancer diagnostic and therapeutic use. The antigen epitope comprises a length of at least 5 consecutive amino acids of SEQ ID NO:1, e.g., 6, 7, 8, 9, 10, 15, 25, 50, 100, 200, 300, 400, 500, 600, 700 or more consecutive amino acids, e.g., the entire TFR1 protein (760 amino acids). Fragments of TFR1 are also useful for any of the above uses; such fragments comprises a length of less than the full-length protein, e.g., a length of at least 5 consecutive amino acids of SEQ ID NO:1, e.g., 6, 7, 8, 9, 10, 15, 25, 50, 100, 200, 300, 400, 500, 600, 700 725, 750, 759 consecutive amino acids (or any length between 5-760 amino acids). TFR1 is a transmembrane protein, and preferably, the TFR1 epitope comprising aberrant glycosylation (compared to a wild type TFR1) is extracellular, i.e., is present on the outside of the cell membrane.

An exemplary method of diagnosing a malignancy is carried out by contacting a bodily tissue or fluid of a subject with an antibody that binds to TFR1, wherein the TFR1 comprises glycosylation at an aberrant site compared to a wild type TFR1 protein. In some embodiments, the method further comprises administering to the subject a cancer therapeutic composition such as a chemotherapeutic agent, immunotherapeutic acid, or radiation therapy. A exemplary subject is characterized as comprising or at risk of developing a colon cancer, a colorectal cancer, a liver cancer, or a lung cancer as well as other tumor types (e.g., adenocarcinoma, bladder cancer, breast cancer, leukemia, lymphoma, pancreatic cancer, thyroid cancer, cancers of the nervous sytem, and colorectal cancer). The bodily fluid or tissue biopsy sample is contacted with the TFR1-specific antibody or other reagent that binds to TFR1 (comprising glycosylation at sites described above) to form a complex between a component of the fluid or tissue sample. The presence of the complex is detected using a fluorescent or other detectable label or radioactive label. The detection of the complex in a patient-derived fluid or tissue sample compared to a normal control sample (or standard level) is indicative of a malignant condition, e.g., the presence of a tumor/cancer in the subject from which the fluid or tissue sample was obtained.

The term “sample” as used herein refers to a biological sample obtained for the purpose of evaluation in vitro. Testing may also be carried out in vivo. With regard to the methods disclosed herein, the sample or patient sample preferably may comprise any body fluid. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In other aspects, the patient-derived sample is a tissue sample, e.g., a biopsy sample. For example, the tissue is colon, colorectal, lung, bladder, breast, pancreatic, thyroid, nervous system, or liver tissue. Presence of the antigen/epitope to which the AF20 antibody binds indicated that the tissue comprises a malignancy, e.g., a tumor.

The invention can alternatively be defined as an improvement over existing methodologies, a method of diagnosing cancer in a subject.

The following definitions are included for the purpose of understanding the present subject matter and for constructing the appended patent claims. Abbreviations used herein have their conventional meaning within the chemical and biological arts.

The term “about” refers to any minimal alteration in the concentration or amount of an agent that does not change the efficacy of the agent in preparation of a formulation and in treatment of a disease or disorder (e.g., cancer). The term “about” with respect to concentration range of the agents (e.g., therapeutic/active agents) of the current disclosure also refers to any variation of a stated amount or range which would be an effective amount or range.

The terms “administration” or “administering” refer to the act of providing an agent of the current embodiments or pharmaceutical composition including an agent of the current embodiments to the individual in need of treatment.

The singular forms “a,” “an,” and “the” include the plural reference unless the context clearly dictates otherwise.

The antibody is a polyclonal antisera or monoclonal antibody. The invention encompasses not only an intact monoclonal antibody, but also an immunologically-active antibody fragment, e. g., a Fab or (Fab)2 fragment; an engineered single chain FV molecule; or a chimeric molecule, e.g., an antibody which contains the binding specificity of one antibody, e.g., of murine origin, and the remaining portions of another antibody, e.g., of human origin.

The invention further comprises a humanized antibody, wherein the antibody is from a non-human species, whose protein sequence has been modified to increase their similarity to antibody variants produced naturally in humans. Generally, a humanized antibody has one or more amino acid residues introduced into it from a source which is non-human. These non-human amino acid residues are referred to herein as “import” residues, which are typically taken from an “import” antibody domain, particularly a variable domain.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

By “fluoroimmunoassay” is meant an assay that has an agent labeled with a fluorophore.

As used herein, the term “nucleic acid” refers to polynucleotides such as deoxyribonucleic acid (DNA, e.g. cDNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

Polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a hhuman subject, e.g., lacking infectious or toxic agents.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the term “salt” refers to acid or base salts of the agents used herein. Illustrative but non-limiting examples of acceptable salts are mineral acid (hydrochloric acid, hydrobromic acid, phosphoric acid, sulfuric acid, and the like) salts, organic acid (acetic acid, propionic acid, glutamic acid, citric acid, and the like) salts, and quaternary ammonium (methyl iodide, ethyl iodide, and the like) salts.

The term “stabilizing agent” refers to a small molecule, an antibody (or fragment thereof), or a binding molecule that yields a complex that does not readily become inactive or denature, for example reversible (e.g., formaldehyde, SPDP (succinimidyl 3-(3-pyridyldithio)propionate)) and non-reversible cross-linkers.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated. With respect to a cell type, an isolated or purified cell is one that has been substantially separated or purified away from other biological components of the organism in which the cell naturally occurs, such as other cells of the organism.

As used herein, “treatment” is an approach for obtaining beneficial or desired results, including clinical results. For purposes of this disclosure, beneficial or desired clinical results include, but are not limited to, alleviation or amelioration of symptoms, diminishment of extent of disease, stabilized (i.e., not worsening) state of disease, preventing spread of disease, delay or slowing of disease progression, amelioration or palliation of the disease state, and recovery (whether partial or total), whether detectable or undetectable. “Treatment” can also mean prolonging survival as compared to expected survival if not receiving treatment.

Insofar as the methods of the present disclosure are directed to compositions and methods for treating a disease or disease state, it is understood that the term “prevent” does not require that the disease state (e.g., cancer) be completely thwarted. The term “prevent” can encompass partial effects when the agents disclosed herein are administered as a prophylactic measure. The prophylactic measures include, without limitation, administration to one (or more) individual(s) who is suspected of being diagnosed with, e.g., cancer.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Other features and advantages of the invention will be apparent from the following description of the preferred embodiments thereof, and from the claims. All references cited herein are hereby incorporated by reference.

### BRIEF DESCRIPTION OF THE DRAWINGS

FIG. 1 is a blot showing the comparison of AF20 antigen levels in different cell lines. Cell lysate containing 200 μg of proteins was subjected to immunoprecipitation with AF20 antibody followed by Western blot using the same antibody. The 50-kDa protein band in the blot corresponds to the heavy chain of AF20 antibody (IgG). Pro RG: proliferating HepaRG cells.

FIG. 2A is an elution profile showing the purification of AF20 antigen from Huh7 cells for proteomic analysis. Cell pellet (2 grams) was lyzed by freeze/thaw and diluted with Tris buffer. The sample was loaded onto a DEAE-cellulose column. After flow through (FT), the column was washed with Tris buffer (wash). Proteins were eluted successively with 100 mM, 200 mM and 400 mM of NaCl, and protein concentration was determined by BCA assay using known concentrations of albumin as a standard.

FIG. 2B is a blot showing that AF20 antigen was imunoprecipitated from the three protein peaks by AF20 mAb using Western blot analysis (minigel format).

FIG. 2C is an SDS-PAGE gel (large gel format) stained with Coomassie blue showing the pooled protein peaks.

FIG. 3A is a blot showing Huh7 cells transiently transfected with DDK tagged TFR1 cDNA. Lysate of transfected or non-transfected cells was immunoprecipitated with DDK or AF20 mAb, followed by sequential detection of the blot with DDK and TFR1 antibodies.

FIG. 3B is a blot showing lysate of nontransfected Huh7 cells incubated with AF20 mAb immobilized on protein G beads, or protein G beads alone to serve as a negative control, followed by Western blot detection of beads-associated proteins by antibodies against AF20, TFR1, HSP and N⁺/K⁺ ATPase, respectively.

FIG. 4 are images showing the affinity of AF20 mAb for TFR1 but not N⁺(K⁺ ATPase or HSP90 by immunofluorescent staining.

FIG. 5A is an image showing that TFR1 transfected NIH 3T3 cells were fixed and stained with either AF20 mAb.

FIG. 5B is an image showing that TFR1 transfected NIH 3T3 cells were fixed and stained with TFR1 mAb.

FIG. 5C is an image showing stacks of X axis for FIG. 5A and 5B, respectively.

FIG. 6A is a blot showing lysate of LS180 cells subjected to immunoprecipitation with AF20 mAb in the absence (w/o) or presence of 33 μg/ml of apo or holo transferrin (TF), and retained proteins were detected by Western blot with AF20 mAb.

FIG. 6B is a blot showing that increasing concentrations (3.3, 16.5, and 66 μg/ml) of holo transferrin added during immunoprecipitation with AF20 mAb, followed by Western blot with the same antibody.

FIG. 7 is a blot showing that AF20 mAb failed to recognize deglycosylated TFR1. Huh7 cell lysate was subject to immunoprecipitation with AF20 mAb and retained proteins on protein G beads were treated with PNGase F at 37° C. for lhr. Samples were directly loaded onto 10% SDS-PAGE (minigel) in duplicate followed by Western blot detection using AF20 (left) and TFR1 antibodies (right), respectively. An additional protein band above the 75-kd size marker was detected by TFR1 antibody from PNGase F treated sample, most likely corresponding to deglycosylated form of TFR1 (79kDd).

FIG. 8 are images of tissue sections showing a correlation of AF20 expression of malignant transformation of colon tissues. A total of 4 pairs of tissue sections were stained with AF20 antibody followed by incubation with HRP-conjugated anti-mouse antibody. Two representative pairs are shown (upper and lower panels). AF20 was clearly detectable in colon polyps (middle panels), and strongly expressed in colon cancer (right panels), but not in normal colon tissue (left panels). Images were taken at 20× magnifications, with the scale bar provided.

FIG. 9A is a tissue section image of a normal colon sample stained with TFR1 (upper panels, 1:50 dilution) and AF20 mAb (lower panels, 1:500 dilution), respectively. Images were taken at 1000× magnification. Overexpression of both TFR1 and AF20 antigen was not observed in normal colon samples. The expression pattern and localization as revealed by TFR1 and AF20 Ab are indistinguishable.

FIG. 9B is a tissue section image of a colon cancer sample stained with TFR1 (upper panels, 1:50 dilution) and AF20 mAb (lower panels, 1:500 dilution), respectively. Images were taken at 1000× magnification. Overexpression of both TFR1 and AF20 antigen was observed in colon cancer samples. The expression pattern and localization as revealed by TFR1 and AF20 Ab are indistinguishable.

FIG. 10 is blot showing Huh7 cells transfected with cDNA encoding DDK-tagged TFR1 or DDK-tagged Na⁺/K⁺-ATPase. Cells were harvested two days later, and cell lysate in duplicate was immunoprecipitated with DDK mAb followed by blotting with either DDK mAb or AF20 mAb. The results suggest that TFR1 (the AF20 antigen) and ATPase could be co-precipitated. The protein band of 75 kd in DDK blot was due to non-specific binding of the antibody

### DETAILED DESCRIPTION OF THE INVENTION

Since the generation of the AF20 monoclonal antibody more than 20 years ago, the molecular identity of its target antigen has remained a longstanding puzzle.

The AF20 antibody was developed after immunizing mice with a HCC cell line (FOCUS) using a schedule designed to generate monoclonal antibodies (mAb) against overexpressed cell surface proteins on the tumor cell line. However, great difficulty was encountered in characterizing the AF20 antigen. The hybridoma producing AF20 was identified, since the antibody highly reacted with human tumors and cell lines derived thereof and included liver, lung, and colon tumors. The antibody did not recognize expression of the antigen on most normal human tissues. Previous attempts to clone the AF20 antigen used a FOCUS HCC cell line derived cDNA library, these attempts were unsuccessful likely because this library was missing the 5′ ends of the cDNA which may have encoded for the AF20 antigen. The molecular weight of the AF20 antigen was determined by metabolic labeling with 5³²-Methionine or direct labeling with 1¹²⁵ followed by immunoprecipitation experiments, which gave a molecular weight of 180 kDa antigen. The early studies demonstrated that it was synthesized as a 90 kDa homodimer and assembled on the cell surface of tumor cells as a 180 kDa protein comprised of two 90 kDa peptides linked by two disulfide bridges. However, numerous attempts to immunoprecipitate the AF20 antigen in tumors and tumor cell lysates were unsuccessful. For nearly 25 years, a number of investigators were unsuccessful in identifying the molecular identity of the AF20 antigen. The invention has provided a solution to this longstanding problem in cancer diagnosis and therapy.

The present work describes and confirms why it was so difficult to identify the antigen precisely. The AF20 epitope on the 180 kDa protein was a post translational modification of glycosylation and was actually in a complex with two other proteins of the identical size. The AF20 antigen is actually on transferrin-1 and is generated by abnormal glycosylation by the machinery operating in tumor tissues. The technique for identifying the 3 proteins that comprise the complex is described herein. The surprising discovery made while identifying the AF20 antigen was that the AF20 antigen is generated during malignant transformation by abnormal glycosylation of this transferrin receptor protein. This discovery was completely unexpected.

The elucidation and purification of this tumor antigen has broad implications for human cancer diagnosis and therapy. The purified antigen and antibodies specific for the antigen has great utility in identification of and treatment of malignancies. For example, it is useful as an imaging agent, as well as a carrier of a drug or isotope since once the antibody binds to the antigen complex, is internalized into the cell. It is also useful as a diagnostic marker of cell transformation such as screening for dysplastic cellular changes in long standing ulcerative colitis which has a high risk of developing colon cancer. It is also used to evaluate prognosis with respect to early tumor reoccurrence and overall survival.

Extensive studies have revealed specific expression of AF20 antigen in human hepatoma and colon cancer cells. The rapid internalization of AF20 antibody in human hepatoma cell lines raised the possibility of specific and highly efficient treatment of liver cancer by conjugating small molecule drugs into the antibody (Mohr L,et al. Gastroenterology 2004;127:S225-231, and Moradpour D et al. Hepatology 1995;22:1527-1537). While immunofluorescent staining of the antigen indicated its cell surface localization, detection by direct Western blot analysis has been unsuccessful. Rather, a combination of immunoprecipitation (IP) and Western blotting was needed.

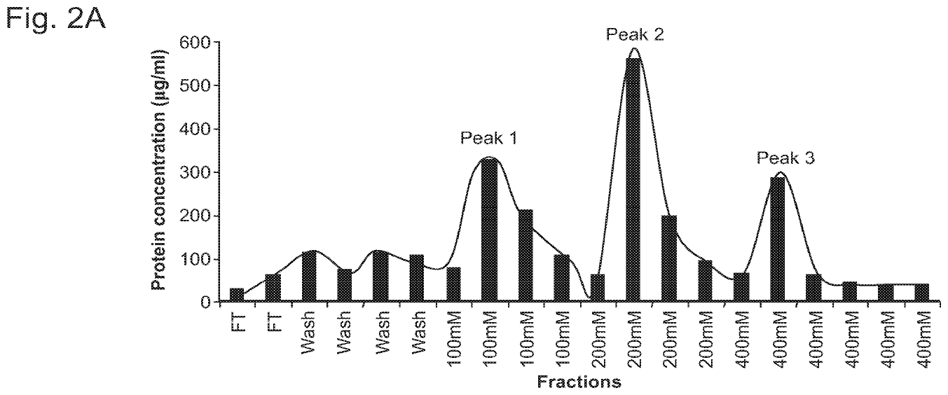

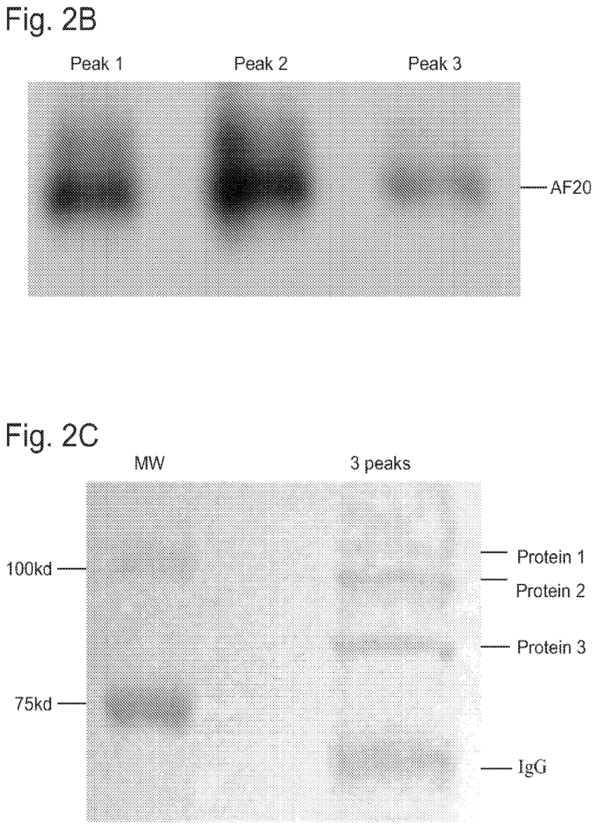

In the present study, IP—SDS PAGE was combined with ion-exchange chromatography to purify the AF20 antigen. Curiously, the AF20 antigen failed to be eluted from DEAE-cellulose column as a single peak. Rather, it was present in eluents of all the three NaCl concentrations suggesting variable affinities for the negatively charged column (FIG. 2B). Peptide sequencing of the tryptic digests revealed two or three proteins instead of a single protein despite further purification by IP—SDS PAGE, which enriched AF20 antigen according to its immunological property as well as molecular size (90-110 kDa).

Consequently, the three proteins identified: TFR1, HSP90, and Na⁺/K⁺ ATPase, shared similar molecular weights. Indeed, in the subsequent reconstitution experiments using cDNA transfection, IP—Western blot analysis revealed ability of the AF20 antibody to immunoprecipitate TFR1, HSP90, as well as Na⁺/K⁺ ATPase. Considering that a monoclonal antibody recognizes an epitope of just 5-7 residues, the AF20 epitope could be present in all the three proteins. The AF20 epitope is present in just one of the three proteins described, and the other two proteins were co-purified with the true AF20 antigen through complex formation.

Surprisingly, the AF-20 epitope was found to be present in TFR1 Two lines of experimental evidence supported the second interpretation and implicated TFR1 [alternatively known as TFR, p90, and CD71 (Tortorella S et al. J Membr Biol 2014;247:291-307)], as the bona fide AF20 antigen. First, transient transfection with TFR1 but not Na⁺/K⁺ ATPase or HSP90 cDNA conferred strong cell surface staining by AF20 antibody in NIH 3T3 cells, which express little endogenous AF20 antigen. Second, holo transferrin could compete for the AF20 antigen - antibody interaction during immunoprecipitation. As TFR1 has much greater affinity for the diferric transferrin (Dautry-Varsat A Proc Nall Acad Sci 1983;80:2258-2262), this finding also indicates the overlap between the transferrin binding site and the AF20 epitope. In addition, known features of TFR1 are also consistent with those of the AF20 antigen. TFR2, on the other hand, is a protein of 355 residues (approximately 39 kDa). Moreover, ability of holo transferrin to interfere with the interaction of AF20 antigen—antibody is also compatible with TFR1, which has much greater affinity for diferric transferrin than TFR2.

### Transferrin Receptor-1

TFR1 is a type II transmembrane protein. Its 760 residues consist of the short cytoplasmic domain (residues 1-67), a single transmembrane domain (residues 68-88), and a large extracellular domain containing three N-linked glycosylation sites. Tunicamycin treatment not only reduced size of TFR1 from 94 kDa to 79 kDa, but also prevented its dimerization. Deglycosylated TFR1 lost affinity for transferrin (Reckhow CL and Enns CA. J Biol Chem 1988;263:7297-7301). The finding that deglycosylated TFR1 is unable to bind AF20 antibody indicates a role of N-linked glycans in recognition by the AF20 mAb. Alternatively, deglycosylation of TFR1 renders the protein unstable to prevent binding by the AF20 mAb.

TFR1 is responsible for iron deposition to most cell types. At neutral pH it has higher affinity for holo transferrin than apo transferrin. Following clathrin-mediated endocytosis, the acidic environment in the endosome triggers iron release from transferrin but increases the affinity of apo transferrin for TFR1 (Dautry-Varsat A et al. Proc Nail Acad Sci 1983;80:2258-2262). After fusion of endosomes with plasma membrane, the neutral pH promotes the release of apo transferrin from TFR1, thus completing the cycle of iron transport. A single cycle takes only 10-20 minutes (Bleil JD and Bretscher MS. EMBO J 1982;1:351-355), which is reminiscent of rapid internalization of the AF20 antibody in human hepatoma cells (Moradpour D, Hepatology 1995;22:1527-1537).

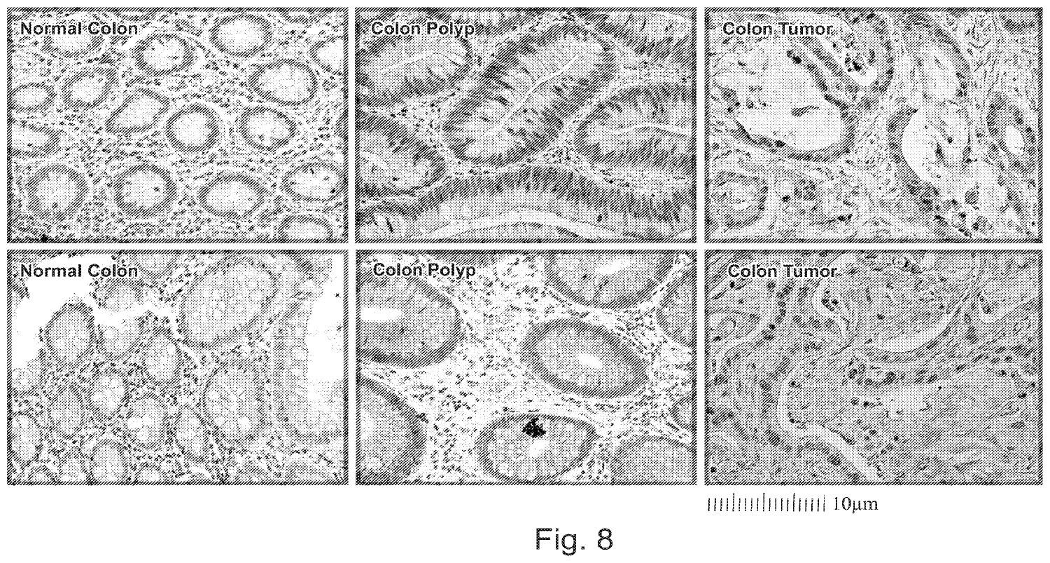

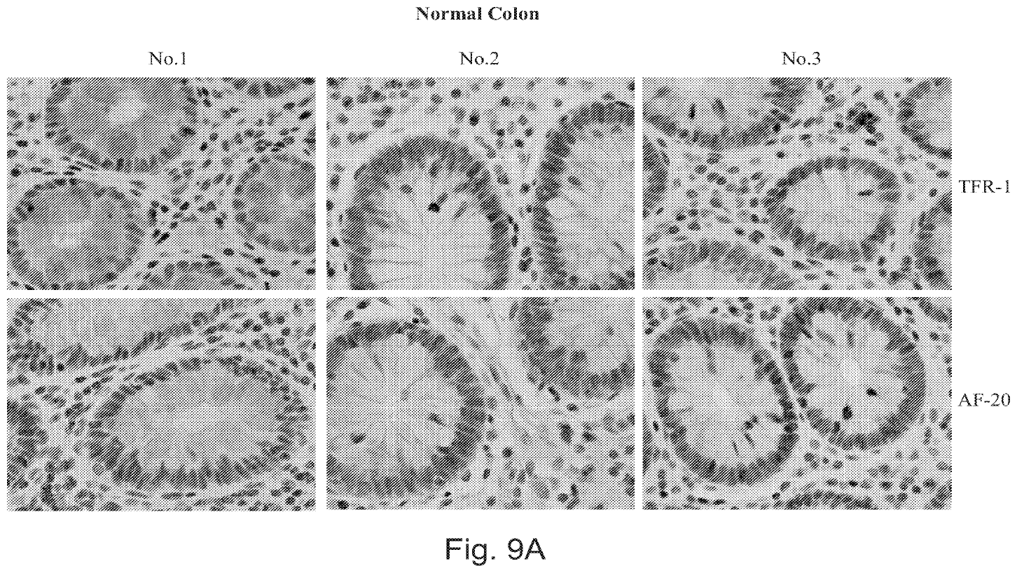

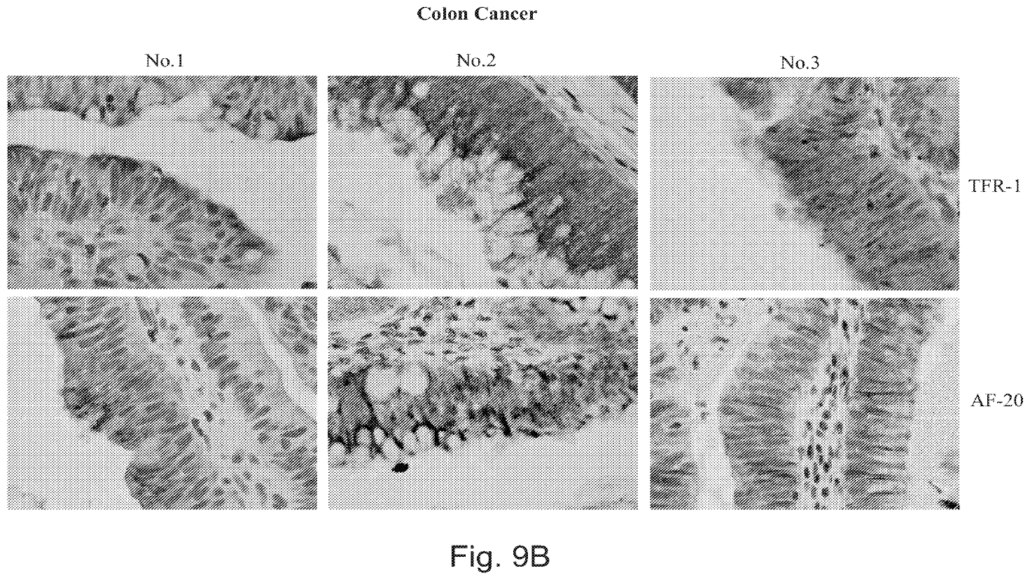

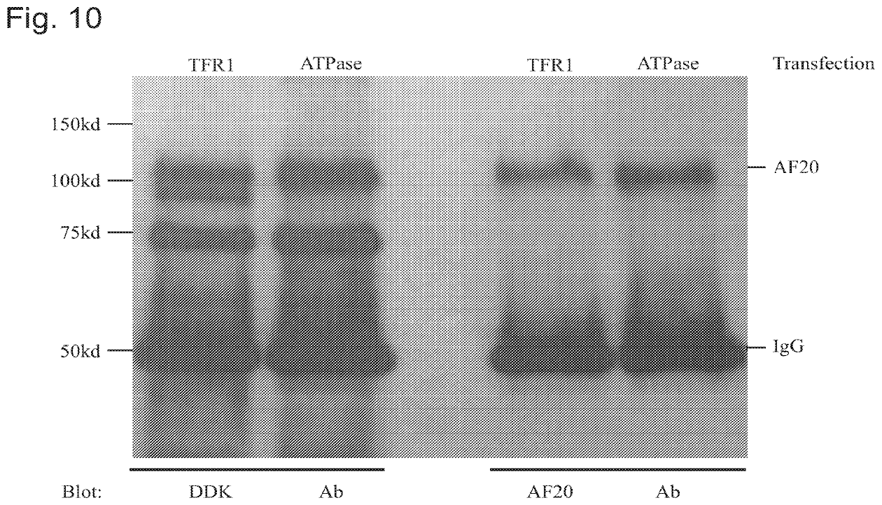

TFR1 is ubiquitously expressed in normal tissues at low level. In the present study, AF20 was undetectable in normal colon but clearly detected in polyps and strongly positive in colon cancer (FIGS. 8 and 9A and 9B).

Human Transferrin Receptor-lamino acid sequence:

```
    1 mmdqarsafs nlfggeplsy trfslarqvd gdnshvemkl avdeeenadn ntkanvtkpk
  61 rcsgsicygt iavivfflig fmigylgyck gvepktecer ragtespvre epgedfpaar
121 rlywddlkrk lsekldstdf tgtikllnen syvpreagsq kdenlalyve nqfrefklsk
181 vwrdqhfvki qvkdsaqnsv iivdkngrlv ylvenpggyv ayskaatvtg klvhanfgtk
241 kdfedlytpv ngsivivrag kitfaekvan aeslnaigvl iymdqtkfpi vnaelsffgh
301 ahlgtgdpyt pgfpsfnhtq fppsrssglp nipvqtisra aaeklfgnme gdcpsdwktd
361 stcrmvtses knvkltvsnv lkeikilnif gvikgfvepd hyvvvgaqrd awgpgaaksg
421 vgtalllkla qmfsdmvlkd gfqpsrsiif aswsagdfgs vgatewlegy lsslhlkaft
481 yinldkavlg tsnfkvsasp llytliektm qnvkhpvtgq flyqdsnwas kvekltldna
541 afpflaysgi pavsfcfced tdypylgttm dtykelieri pelnkvaraa aevagqfvik
601 lthdvelnld yerynsqlls fvrdlnqyra dikemglslq wlysargdff ratsrlttdf
661 gnaektdrfv mkklndrvmr veyhflspyv spkespfrhv fwgsgshtlp allenlklrk
721 qnngafnetl frnqlalatw tiqgaanals gdvwdidnef
```

(SEQ ID NO: 1) GenBank Accession NP_001121620 version 1, incorporated herein by reference.

Exemplary regions or fragments of Transferrin Receptor-linclude residues 1-67, 20-23 (endocytosis signal), 68-88 (transmembrane region), 100-101, 101-760, 201-377, 385-610, 569-760, 642-750, and 646-648 as well as immunogenic fragments thereof for use in immunotherapy. The region may include the cytoplasmic domain (residues 1-67), or the extracellular domain (residues 89-763). Furthermore, the region may include the ligand-binding region (residues 572-763), the endocytosis signal (residues 20-23), the stop-transfer sequence (residues 58-61), the apical domain (residues 324-368), and the cell attachment site residues 649-651. In some examples, the fragment comprises of a cell surface epitope. Exemplary peptides or fragments of Transferrin Receptor 1 include those which comprise an N-linked glycosylation site, e..g, as described in Medzihradszky K. F., Methods Mol Biol. 2008;446:293-316; hereby incorporated by reference. N-glycosylated proteins are modified at Asn residues. There is a consensus sequence for N-glycosylation: AsnXxxSer/Thr/Cys, where Xxx can be any amino acid except proline. For example, wild type TFR-1 includes N-linked glycosylation sites at amino acid positions 251, 317, and 727 of SEQ ID NO:1 (Williams et al., 1993, J. Biol. Chem. 268(17): 12780-12786; Daniels et al., 2012, Biochim Biophys Acta. 1820(3): 291-317; each of which is hereby incorporated by reference. N-linked glycosylation sites are also present at amino acids 50 and 55 (N50 and/or N55) of SEQ ID NO:1. An O-linked glycosylation site is present at amino acid position 104 (T104) of SEQ ID NO:1

A fragment has a length that is less than the length of the full-length reference peptide. For example, in the case of the transferrin receptor 1 shown above, a fragment is a peptide that containg greater than 1 amino acid and contains less than 760 amino acids, e.g., the fragment contains or contains less than 5, 8, 9, 10, 12, 15, 20, 25, 30, 40, 50, 75, 100, 200, 300, 400, 500, 600, 700, or 750 contiguous amino acids of SEQ ID NO: 1. For example, a fragment comprises a TFR1-sprefic antibody-binding epitope of 5-7 amino acids.

Human Transferrin Receptor-1 nucleotide sequence.

```
      1 acgcacagcc cccctggggg ccgggggcgg ggccaggcta taaaccgccg gttaggggcc
    61 gccatcccct cagagcgtcg ggatatcggg tggcggctcg ggacggagga cgcgctagtg
  121 ttcttctgtg tggcagttca gaatgatgga tcaagctaga tcagcattct ctaacttgtt
  181 tggtggagaa ccattgtcat atacccggtt cagcctggct cggcaagtag atggcgataa
  241 cagtcatgtg gagatgaaac ttgctgtaga tgaagaagaa aatgctgaca ataacacaaa
  301 ggccaatgtc acaaaaccaa aaaggtgtag tggaagtatc tgctatggga ctattgctgt
  361 gatcgtcttt ttcttgattg gatttatgat tggctacttg ggctattgta aaggggtaga
  421 accaaaaact gagtgtgaga gactggcagg aaccgagtct ccagtgaggg aggagccagg
  481 agaggacttc cctgcagcac gtcgcttata ttgggatgac ctgaagagaa agttgtcgga
  541 gaaactggac agcacagact tcaccggcac catcaagctg ctgaatgaaa attcatatgt
  601 ccctcgtgag gctggatctc aaaaagatga aaatcttgcg ttgtatgttg aaaatcaatt
  661 tcgtgaattt aaactcagca aagtctggcg tgatcaacat tttgttaaga ttcaggtcaa
  721 agacagcgct caaaactcgg tgatcatagt tgataagaac ggtagacttg tttacctggt
  781 ggagaatcct gggggttatg tggcgtatag taaggctgca acagttactg gtaaactggt
  841 ccatgctaat tttggtacta aaaaagattt tgaggattta tacactcctg tgaatggatc
  901 tatagtgatt gtcagagcag ggaaaatcac ctttgcagaa aaggttgcaa atgctgaaag
  961 cttaaatgca attggtgtgt tgatatacat ggaccagact aaatttccca ttgttaacgc
1021 agaactttca ttctttggac atgctcatct ggggacaggt gacccttaca cacctggatt
1081 cccttccttc aatcacactc agtttccacc atctcggtca tcaggattgc ctaatatacc
1141 tgtccagaca atctccagag ctgctgcaga aaagctgttt gggaatatgg aaggagactg
1201 tccctctgac tggaaaacag actctacatg taggatggta acctcagaaa gcaagaatgt
1261 gaagctcact gtgagcaatg tgctgaaaga gataaaaatt cttaacatct ttggagttat
1321 taaaggcttt gtagaaccag atcactatgt tgtagttggg gcccagagag atgcatgggg
1381 ccctggagct gcaaaatccg gtgtaggcac agctctccta ttgaaacttg cccagatgtt
1441 ctcagatatg gtcttaaaag atgggtttca gcccagcaga agcattatct ttgccagttg
1501 gagtgctgga gactttggat cggttggtgc cactgaatgg ctagagggat acctttcgtc
1561 cctgcattta aaggctttca cttatattaa tctggataaa gcggttcttg gtaccagcaa
1621 cttcaaggtt tctgccagcc cactgttgta tacgcttatt gagaaaacaa tgcaaaatgt
1681 gaagcatccg gttactgggc aatttctata tcaggacagc aactgggcca gcaaagttga
1741 gaaactcact ttagacaatg ctgctttccc tttccttgca tattctggaa tcccagcagt
1801 ttctttctgt ttttgcgagg acacagatta tccttatttg ggtaccacca tggacaccta
1861 taaggaactg attgagagga ttcctgagtt gaacaaagtg gcacgagcag ctgcagaggt
1921 cgctggtcag ttcgtgatta aactaaccca tgatgttgaa ttgaacctgg actatgagag
1981 gtacaacagc caactgcttt catttgtgag ggatctgaac caatacagag cagacataaa
2041 ggaaatgggc ctgagtttac agtggctgta ttctgctcgt ggagacttct tccgtgctac
2101 ttccagacta acaacagatt tcgggaatgc tgagaaaaca gacagatttg tcatgaagaa
2161 actcaatgat cgtgtcatga gagtggagta tcacttcctc tctccctacg tatctccaaa
2221 agagtctcct ttccgacatg tcttctgggg ctccggctct cacacgctgc cagctttact
2281 ggagaacttg aaactgcgta aacaaaataa cggtgctttt aatgaaacgc tgttcagaaa
2341 ccagttggct ctagctactt ggactattca gggagctgca aatgccctct ctggtgacgt
2401 ttgggacatt gacaatgagt tttaaatgtg atacccatag cttccatgag aacagcaggg
2461 tagtctggtt tctagacttg tgctgatcgt gctaaatttt cagtagggct acaaaacctg
2521 atgttaaaat tccatcccat catcttggta ctactagatg tctttaggca gcagctttta
2581 atacagggta gataacctgt acttcaagtt aaagtgaata accacttaaa aaatgtccat
2641 gatggaatat tcccctatct ctagaatttt aagtgctttg taatgggaac tgcctctttc
2701 ctgttgttgt taatgaaaat gtcagaaacc agttatgtga atgatctctc tgaatcctaa
2761 gggctggtct ctgctgaagg ttgtaagtgg tcgcttactt tgagtgatcc tccaacttca
2821 tttgatgcta aataggagat accaggttga aagaccttct ccaaatgaga tctaagcctt
2881 tccataagga atgtagctgg tttcctcatt cctgaaagaa acagttaact ttcagaagag
2941 atgggcttgt tttcttgcca atgaggtctg aaatggaggt ccttctgctg gataaaatga
3001 ggttcaactg ttgattgcag gaataaggcc ttaatatgtt aacctcagtg tcatttatga
3061 aaagagggga ccagaagcca aagacttagt atattttctt ttcctctgtc ccttccccca
3121 taagcctcca tttagttctt tgttattttt gtttcttcca aagcacattg aaagagaacc
3181 agtttcaggt gtttagttgc agactcagtt tgtcagactt taaagaataa tatgctgcca
3241 aattttggcc aaagtgttaa tcttagggga gagctttctg tccttttggc actgagatat
3301 ttattgttta tttatcagtg acagagttca ctataaatgg tgttttttta atagaatata
3361 attatcggaa gcagtgcctt ccataattat gacagttata ctgtcggttt tttttaaata
3421 aaagcagcat ctgctaataa aacccaacag atactggaag ttttgcattt atggtcaaca
3481 cttaagggtt ttagaaaaca gccgtcagcc aaatgtaatt gaataaagtt gaagctaaga
3541 tttagagatg aattaaattt aattaggggt tgctaagaag cgagcactga ccagataaga
3601 atgctggttt tcctaaatgc agtgaattgt gaccaagtta taaatcaatg tcacttaaag
3661 gctgtggtag tactcctgca aaattttata gctcagttta tccaaggtgt aactctaatt
3721 cccattttgc aaaatttcca gtacctttgt cacaatccta acacattatc gggagcagtg
3781 tcttccataa tgtataaaga acaaggtagt ttttacctac cacagtgtct gtatcggaga
3841 cagtgatctc catatgttac actaagggtg taagtaatta tcgggaacag tgtttcccat
3901 aattttcttc atgcaatgac atcttcaaag cttgaagatc gttagtatct aacatgtatc
3961 ccaactccta taattcccta tcttttagtt ttagttgcag aaacattttg tggtcattaa
4021 gcattgggtg ggtaaattca accactgtaa aatgaaatta ctacaaaatt tgaaatttag
4081 cttgggtttt tgttaccttt atggtttctc caggtcctct acttaatgag atagtagcat
4141 acatttataa tgtttgctat tgacaagtca ttttaacttt atcacattat ttgcatgtta
4201 cctcctataa acttagtgcg gacaagtttt aatccagaat tgaccttttg acttaaagca
4261 gagggacttt gtatagaagg tttgggggct gtggggaagg agagtcccct gaaggtctga
4321 cacgtctgcc tacccattcg tggtgatcaa ttaaatgtag gtatgaataa gttcgaagct
4381 ccgtgagtga accatcatta taaacgtgat gatcagctgt ttgtcatagg gcagttggaa
4441 acggcctcct agggaaaagt tcatagggtc tcttcaggtt cttagtgtca cttacctaga
4501 tttacagcct cacttgaatg tgtcactact cacagtctct ttaatcttca gttttatctt
4561 taatctcctc ttttatcttg gactgacatt tagcgtagct aagtgaaaag gtcatagctg
4621 agattcctgg ttcgggtgtt acgcacacgt acttaaatga aagcatgtgg catgttcatc
4681 gtataacaca atatgaatac agggcatgca ttttgcagca gtgagtctct tcagaaaacc
4741 cttttctaca gttagggttg agttacttcc tatcaagcca gtacgtgcta acaggctcaa
4801 tattcctgaa tgaaatatca gactagtgac aagctcctgg tcttgagatg tcttctcgtt
4861 aaggagatgg gccttttgga ggtaaaggat aaaatgaatg agttctgtca tgattcacta
4921 ttctagaact tgcatgacct ttactgtgtt agctctttga atgttcttga aattttagac
4981 tttctttgta aacaaatgat atgtccttat cattgtataa aagctgttat gtgcaacagt
5041 gtggagattc cttgtctgat ttaataaaat acttaaacac tgaaaaaaaa aaa
```

(SEQ ID NO: 2) GenBank Accession NM_001128148, version 2 incorporated herein by reference.

Exemplary regions or fragments of Transferrin Receptor-1 nucleic acid sequences include residues 197-199, 200-211, 314-325, 344-406, 1847-2422, 2078-2086, 830-943, 944-1042, and 4932-5057.

### AF-20

AF-20 antibody is a monoclonal antibody and is an anti-hepatocellular carcinoma (HCC) monoclonal antibody that binds to a rapidly internalized 180-kDa homodimeric glycoprotein present in high amounts on the surface membrane of human HCC and other human cancer cell lines.

Immunizing mice with hepatoma cells derived from an HCC cell line (FOCUS), a handful of monoclonal antibodies were isolated with high affinity to cancer cells but not to normal or non-transformed hepatocytes (Wilson B et al. Proc Natl Acad Sci 1988;85:3140-4). One of the antibodies, namely AF20, recognized a protein of apparent molecular size of 90-110 kDa. The AF20 antigen was found abundantly expressed on cell surface of human HCC cell lines, as well as human colon cancer cell lines such as LS180 and HT-29 (Mohr L et al. Gastroenterology 2004;127:5225-231, and Moradpour D et al. Hepatology 1995;22:1527-1537). Binding of AF20 antibody to the cancer cells was followed by its rapid internalization, which raises the hope of specific and highly efficient delivery of therapeutic drugs or small molecules into cancer cells (Moradpour D et al. Hepatology 1995;22:1527-1537, and Wands J R, et al. J Viral Hepat 1997;4 Suppl 2:60-74). In vivo experiments validated AF20's ability to differentiate human HCC from adjacent normal liver tissue (Wilson B, et al. Proc Natl Acad Sci 1988;85:3140-4). AF20 serves as a biomarker for early detection and diagnosis of malignant transformation, and also as a vehicle for delivery of anti-tumor drugs with high affinity and specificity. Further characterization revealed AF20 antigen as a dimer of 90-kDa glycoprotein linked together by disulfide bonds (Moradpour D et al. Hepatology 1995;22:1527-1537). The AF20 antigen is identical to the glycosylated form of human transferrin receptor 1 (TFR1), which can form a protein complex with heat shock protein 90 (HSP90) and/or transporting ATPase

### Human AF-20

AF20 monoclonal antibody (mAb) was produced and characterized as previously described (Wilson B et al. Proc Natl Acad Sci 1988;85:3140-4 , Moradpour D et al. Hepatology 1995;22:1527-1537, and Wands J R, et al. J Viral Hepat 1997;4 Suppl 2:60-74), incorporated herein by reference.

Heat shock protein 90 (Hsp90)

Hsp90 is a chaperone protein that assists other proteins to fold properly, stabilizes proteins against heat stress, and aids in protein degradation. It also stabilizes a number of proteins required for tumor growth. Heat shock proteins protect cells when stressed by elevated temperatures and when cells are heated, the fraction of heat shock proteins increases to 4-6% of cellular proteins. Heat shock protein 90 (Hsp90) is one of the most common of the heat-related proteins. The “90” comes from the fact that it weighs roughly 90 kDa.

Heat shock protein 90 (Hsp90) amino acid sequence:

```
    1 mesltdpskl dsgkephisl ipnkqdrtlt ivdtgigmtk adlinnlgti tksetkvfme
  61 vlqagadism igqfsvgfys aysvaekvtv itkhnndeqy awesslrgsf teyrefyksl
121 tinwedylav khfsvegqle fraflfvprl apfelletrk kknkiklsar rdlimdncee
181 lipeylnfir gvvdsedlpl nifretkdqv anstivqrlw khgleviyti epideycvqq
241 lkefegktlv svtkedlelp edeeekkkqe egkqktkqkk nqslrtsaks tygwtanmer
301 imkaqalrdn sttgymaakk hleinpdhsf idtlrqkaet dkndksvkdl villyetall
361 ssdfglegpq thanriyrmn klglgtdedd ptaddtsaav teempplegd ddtsrmek
```

(SEQ ID NO: 5) GenBank Accession Q58FG1 version 1, incorporated herein by reference. Exemplary regions or fragments of Heat shock protein 90 (Hsp90) include residues 11-101, 2-418, and 112-410.

Heat shock protein 90 (Hsp90) nucleotide sequence:

```
      1 gagctccggc tgccctgcac tggttcccag agactccctc cttcccaggt ccaaatggct
    61 gcaggagcga agtgggcgga aaaaaagcga accagcttga gaaagggctt gacgtgcctg
  121 cgtagggagg gcgcatgtcc ccgtgctccg tgtacgtggc ggccgcaggg gctagagggg
  181 ggtccccccc gcaggtactc cactctcagt ctgcaaaagt gtacgcccgc agagccgccc
  241 caggtgcctg ggtgttgtgt gattgacgcg gggaaggagg ggtcagccga tccctcccca
  301 accctccatc ccatccctga ggattgggct ggtacccgcg tctctcggac aggtcagagc
  361 gggtcgccgg gtggggtcgc tgcaaaaacc ctgccccggc cgcagccgag aggcggacgt
  421 cgcggggagg gggcgggacc gccgagacag gcctggaaac tgctggaaat gccgcagtgc
  481 cgccgccgcc ccttccgccg catgtcggca aagagtcccc gccagccccg gccggcgccc
  541 tccccctacg ctgagctgcc cctcagcgcg aaccctccgc ccttcctcta ctcctgcgag
  601 agtcgggatc tggggctacc caaggttggg tcccgaatgc cagtccctct gtcgggacgc
  661 gagatgtgta gggcagatgc taggaagaag attgggtctg ggacgggtgg tccgcgtggt
  721 tagctgcctc cgctcttttt cggtgtcccc cccagtcccg cccttgggtg tggggacgcc
  781 tgccccacaa gtgtttaggg aggtcagtgg gttcctcgcc cgtagagaca ccgtttatgc
  841 caaatgagca ctcctcatcc ccgctcttga tggagtcatg tcctagacgt gaaactatgg
  901 ggctgtgatc acaagcaaat gtgtgggcgg atccgttgct tgggttcttc cccgccccct
  961 ctttttttcg gaccatgacg tcaaggtggg ctggtggcgg caggtgcggg gttgacaatc
1021 atactccttt aaggcggagg gatctacagg agggcggctg tactgtgctt cgccttatat
1081 agggcgactt ggggcacgca gtagctctct cgagtcactc cggcgcagtg ttgggactgt
1141 ctgggtatcg gaaagcaagc ctacgttgct cactattacg tataatcctt ttcttttcaa
1201 ggtaaggctg agatctccgc taggcttctt tccctttagt gctgtattcg tgttgttttt
1261 gtttttttct gtcctttagg gagccttagt ctagatgtcg gggtggcttg tggataacgc
1321 tctggatttt tatagggtga gggtagtggt gggtgaggtt ttttgagtcc tcctcggttt
1381 tctctagtgt gtttgggggg tggggctttc tctcggcgcc tgctggccgt agcgaggtgg
1441 gctgtggggt tggggcagtg ggcggctggc agctgcacgt ggtggccgcg cggcccggga
1501 cgctgccatt tttgcccctc cacttccgga cgcggctacg gggcgtcgga gggggaccgc
1561 aggtggcggg ggtgcccgct cgggtgactc agcacggcct tgtgggactg gctttgtcac
1621 ctctcttatc ggacgcgttg ttaaagcctt cttgggtgct ttgtttctgt gagggagggt
1681 tgacggtgtg ggaagagagc tttcggtctc cagcacccga tactccctcc ttccagatct
1741 ttcttgcagt cccggtggag gaggggcggg gaggggagca ggttctggaa gattcatggg
1801 ctccttcctc cgcccttcct cgagagctga gattgttctg gaagcttctg gattctggcg
1861 ccccgcccca gtgcccggat gctgggggcg agggagggtg cactgcggcg ccccctcctc
1921 gcgtggtcct ggccgacgca tgtccggcag tgacgagtgt cggcctggtg gctacggcca
1981 ccatctttct tgggtttggt cctgttctgt aattttgtgc tgtgaaaggg tcgtggtgga
2041 gcttttggct taagaattct ttgtccggat ttaattgctc ctccggtggg tatcgtatgg
2101 atcccaggtt attcctccct gccctatggg caggagtgtc ccgcccttgg actggtctta
2161 ggaactgaca cctcaggggg agcagtttaa agttagtgcc atttttatct taaactagtc
2221 actttgacct cccccaaata aagaactgta ggtagtgatt ttcacattta aatttgtgta
2281 aggattactt gggatctcta gatacctggg ttggaccaac attatgattt ttctgccata
2341 ctaccagatg atgctgaggc tgctggtcac cattctttaa gtaggtgggt tctgtgacat
2401 ttggttgaag aatatttagc ttattttctt tttccttctg aattttcagg cctcccactt
2461 agtgtgtagt ctgagatctt taagagaatg catttttagt cttgggaagg gatagtactc
2521 cggttaaacc agtctgaact cactgtctaa ggtcctaaca aatgatatga cctttaggat
2581 ttttaaacat ggggccttag tgttcttttg taattaatga gatttttatt ttagatgcct
2641 gaggaagtgc accatggaga ggaggaggtg gagacttttg cctttcaggc agaaattgcc
2701 caactcatgt ccctcatcat caataccttc tattccaaca aggagatttt ccttcgggag
2761 ttgatctcta atgcttctga tgtaggtgct ctggtttcca catttggcat ggtttttttt
2821 tttgatactc tagaaggagg ggaaaggagt ggtttggcct ttgttgggga ctactattga
2881 agggggtaaa cttgcagcta ttccaaaaag atgggtttta ctctggccat cttgaacttg
2941 gaagggacta tgtccagaat aagtgggctc atggaactaa ctggttctaa agcctcaaga
3001 tagggggcaa tcagatttga ggctgagaga ggtaaaccaa gattttcttt gaagatacgg
3061 gctttaagaa agcaaaagtg gctgagcgtg ttggctcaca cctgtaatgc caaggcagga
3121 ggatcacttg agcctaggat ttcgagggca gcctgggcaa ccgcgagacc ttgtctctgc
3181 aaaaaattaa atattagttg ggcacggtgg catgtgctgt agtcccagct acttgggaag
3241 ctggggttgg gaggatggct cgaacctggg aggtcaaggc tgcagtgagc tgtcatcctt
3301 gccactgcac tgtagcttgg gcaacagagc aagagtctgt cttggaaaga gcaaaagtaa
3361 gttgctgttt gtatttccag gccttggaca agattcgcta tgagagcctg acagaccctt
3421 cgaagttgga cagtggtaaa gagctgaaaa ttgacatcat ccccaaccct caggaacgta
3481 ccctgacttt ggtagacaca ggcattggca tgaccaaagc tgatctcata aataatttgg
3541 gaaccattgc caagtctggt actaaagcat tcatggaggc tcttcaggta ttgcagttct
3601 gtaggcattc atacttatct gtgttctttg gttttttgct tctttaaaac ttgtgattga
3661 ctttaaactt gttggcaggc tggtgcagac atctccatga ttgggcagtt tggtgttggc
3721 ttttattctg cctacttggt ggcagagaaa gtggttgtga tcacaaagca caacgatgat
3781 gaacagtatg cttgggagtc ttctgctgga ggttccttca ctgtgcgtgc tgaccatggt
3841 aagttagctt ttctgttaca aggtagttgg gttaggattt tctgggctca caccagtagc
3901 agaaattttg ggcatcctgt ctgtaaagca gttcttcaca gcagttctgc tgatacttac
3961 taattgctgg tctcaactgc atatactttt taccctgtta cacgcttgta attgactctt
4021 ctaggtgagc ccattggcag gggtaccaaa gtgatcctcc atcttaaaga agatcagaca
4081 gagtacctag aagagaggcg ggtcaaagaa gtagtgaaga agcattctca gttcataggc
4141 tatcccatca ccctttatgt gagtatggac ttttaaatct tttacactta acgtgcagga
4201 tgtttcctgt tctggagaat ctcattgtcc ctggcttttg ctttccctgg tagtgttttg
4261 tactccaagg ctaacttctg tttttgttac ttagttggag aaggaacgag agaaggaaat
4321 tagtgatgat gaggcagagg aagagaaagg tgagaaagaa gaggaagata aagatgatga
4381 agaaaaaccc aagatcgaag atgtgggttc agatgaggag gatgacagcg gtaaggataa
4441 gaagaagaaa actaagaaga tcaaagagaa atacattgat caggaagaac taaacaagac
4501 caagcctatt tggaccagaa accctgatga catcacccaa gaggagtatg gagaattcta
4561 caagagcctc actaatgact gggaagacca cttggcagtc aaggtgtgag aagcctttgc
4621 atgttggctc aacatgcaca tatggagagg aatgagttag gtggaagagt gttgggtaat
4681 agacacacgg aacttgtcca actgataaca gaaatgtgat agccatgtga tttcacttac
4741 tgattaccct gtcatagtga agtgccatca tttctaatga cctcactttc tcttcttatg
4801 gaaatctggg taatgtctat tggcagcctt acacatccag ggttctgatc agaggggact
4861 gttttctaca tacagctagt acccatctag atcgtggagg gcattaaggc tcagttttct
4921 caggagctgc ttgttgtgtg tgctctatcc cttaggctta gggaggatca ttgttccact
4981 tttagataat ttggtgttgg ggctaaaagg tcctcttttg aaatgtacca cttatttttg
5041 gtttctttca gcacttttct gtagaaggtc agttggaatt cagggcattg ctatttattc
5101 ctcgtcgggc tccctttgac ctttttgaga acaagaagaa aaagaacaac atcaaactct
5161 atgtccgccg tgtgttcatc atggacagct gtgatgagtt gataccagag tatctcagtg
5221 agtatctcct tggcctaatt tagttgggtg aagtcttggg aggttttagg cattctgcta
5281 ggatattcta aggtaacagt tttctgcaat acatagtagg tgtaagggtt caggaggcta
5341 ttagagcctt ctgtttgaat ctggggacca ggtctggtct agctgttttt actgagcttt
5401 ctcaccctgg ttgatggcag attttatccg tggtgtggtt gactctgagg atctgcccct
5461 gaacatctcc cgagaaatgc tccagcagag caaaatcttg aaagtcattc gcaaaaacat
5521 tgttaagaag tgccttgagc tcttctctga gctggcagaa gacaaggaga attacaagaa
5581 attctatgag gcattctcta aaaatctcaa ggtaaaaagg caaataatgc ttattccctt
5641 taccactttc ttagtaataa caataaatta ttccattcac attgaaagtg aagttattgt
5701 agttaagctg gattgttttt cctcttccca cccttcaagc ttggaatcca cgaagactcc
5761 actaaccgcc gccgcctgtc tgagctgctg cgctatcata cctcccagtc tggagatgag
5821 atgacatctc tgtcagagta tgtttctcgc atgaaggaga cacagaagtc catctattac
5881 atcactggtg cgttgactct gattgaagcc tttttggagg agtggggagc acaattaggg
5941 cttcctggga actggcagta tgaggcattt tagtcactga gttcatttaa ttaccctaca
6001 ggtgagagca aagagcaggt ggccaactca gcttttgtgg agcgagtgcg gaaacggggc
6061 ttcgaggtgg tatatatgac cgagcccatt gacgagtact gtgtgcagca gctcaaggaa
6121 tttgatggga agagcctggt ctcagttacc aaggagggtc tggagctgcc tgaggatgag
6181 gaggagaaga agaagatgga agagagcaag gcaaagtttg agaacctctg caagctcatg
6241 aaagaaatct tagataagaa ggttgagaag gtaagccatt ctggggctag gatatatttt
6301 gtaacatctt cgaggtgggc tccctcacaa gcatgtttct atacaattag tggtttgagg
6361 cagcctattt actgtttcat gccttcttgc ctcttgttct cttctctagt caggtttaag
6421 gctattttaa taaaatttgg cacagattag gcattgcttc agttaacttc tgagagtaga
6481 taaaatacca tcattttctt ttttttttct ttttttgaga tggggtctcg ctctgtcacc
6541 caggctggag tgcagtggca cgatctctgc tcattgcaag ctccgcctcc tgggttcacg
6601 ccattctcct gccttagcct cctgagtagc tgccactaca ggcgcccgcc accacacccc
6661 ggctaatttt ttgtattttt agtagagatg gggtttcatc gcgttagcca ggatggtctc
6721 catctcctga ccttgtgatt cgcccacctc ggcctcccaa agtgctggga ttaacaggcg
6781 caagccacca tgcctggccg attttttttt tttttggact ggatctcgct cactgcaaac
6841 tcaagtctcc tgagtggctg ggattacaga tgtgtgctac cacacccggt taattttttg
6901 tagacagggt tttgccatgt tggccagcat ggtctcaaac tcaagtggtc tgtccacctc
6961 ctccccctgc tggaattagg cttgacaatg cctgttttct ctttcaaagt ggtaatgtca
7021 atctaaggct tttgtgatcg tccacaggtg acaatctcca atagacttgt gtcttcacct
7081 tgctgcattg tgaccagcac ctacggctgg acagccaata tggagcggat catgaaagcc
7141 caggcacttc gggacaactc caccatgggc tatatgatgg ccaaaaagca cctggagatc
7201 aaccctgacc accccattgt ggagacgctg cggcagaagg ctgaggccga caagaatgat
7261 aaggcagtta aggacctggt ggtgctgctg tttgaaaccg ccctgctatc ttctggcttt
7321 tcccttgagg atccccagac ccactccaac cgcatctatc gcatgatcaa gctaggtcta
7381 ggtaagtagc tttggtactt ggtgtggcaa ggagtttgtg caactcgtct cctctatgga
7441 tttgacttaa tgctatttgg tcaagtctca catggcttaa ttttacttca ggtattgatg
7501 aagatgaagt ggcagcagag gaacccaatg ctgcagttcc tgatgagatc ccccctctcg
7561 agggcgatga ggatgcgtct cgcatggaag aagtcgatta ggttaggagt tcatagttgg
7621 aaaacttgtg cccttgtata gtgtccccat gggctcccac tgcagcctcg agtgcccctg
7681 tcccacctgg ctccccctgc tggtgtctag tgtttttttc cctctcctgt ccttgtgttg
7741 aaggcagtaa actaagggtg tcaagcccca ttccctctct actcttgaca gcaggattgg
7801 atgttgtgta ttgtggttta ttttattttc ttcattttgt tctgaaatta aagtatgcaa
7861 aataaagaat atgccgtttt tatacagttc tgctttccct tgtgaagtgg atgttatcct
7921 tccctagctt cttcatccct ccagctcttg ctgttttcat gagcacagca agttgagctg
7981 gttttgtagt gaaaataaca gaataccagt gagtcttaag agttcacaca ctgaagctaa
8041 aggcagtttg gaaaaactac catataataa tgccctttca gtcaaccaaa acacaggacc
8101 aagtccactg cagtaattta atttaataaa ataaaattat aagagcaaaa agttacattt
8161 ctaaagtacc aaaacctgca acaggctcat ggaacagagc ctagggatcc
```

(SEQ ID NO: 6) GenBank Accession J04988 version 1, incorporated herein by reference. Exemplary regions or fragments of Heat shock protein 90 (Hsp90) include bases 453-466, 463-476, 567-580, 1103-7886, 1202-2634, 2434-2450, 5740-5887, and 7382-7491.

ATPase (Na⁺/K⁺ or Mg⁺⁺)

ATPases are a class of enzymes that catalyze the decomposition of ATP into ADP and a free phosphate ion. This dephosphorylation reaction releases energy, which the enzyme (in most cases) harnesses to drive other chemical reactions that would not otherwise occur. Some such enzymes are integral membrane proteins (anchored within biological membranes), and move solutes across the membrane, typically against their concentration gradient. These are called transmembrane ATPases. Transmembrane ATPases import many of the metabolites necessary for cell metabolism and export toxins, wastes, and solutes that can hinder cellular processes. An important example is the sodium-potassium exchanger (or Na⁺/K⁺ ATPase) that maintains the cell membrane potential. And another example is the hydrogen potassium ATPase (H⁺/K⁺ ATPase or gastric proton pump) that acidifies the contents of the stomach. Besides exchangers, other categories of transmembrane ATPase include co-transporters and pumps (however, some exchangers are also pumps). Some of these, like the Na⁺/K⁺ ATPase, cause a net flow of charge, but others do not.

Na⁺/K⁺-ATPase amino acid sequence:

```
    1 mviqkekksc gqvveewkef vwnprthqfm grtgtswafi llfylvfygf ptamftltmw
  61 vmlqtvsdht pkyqdrlatp glmirpkten ldvivnvsdt eswdqhvqkl nkflepynds
121 mqaqkndvcr pgryyeqpdn gvlnypklac qfnrtqlgnc sgigdsthyg ystgqpcvfi
181 kmnrvinfya ganqsmnvtc agkrdedaen lgnfvmfpan gnidlmyfpy ygkkfhvnyt
241 qplvavkfln vtpnvevnve crinaaniat dderdkfagr vafklrinkt
```

(SEQ ID NO: 7) GenBank Accession AAA51805 version 1, incorporated herein by reference. Exemplary regions or fragments of Na±/K+-ATPase include residues 1-290 and 2-289.

Na⁺/K⁺-ATPase nucleotide sequence:

```
      1 gaattcatgc taaattgctg gaaggctgcg tctctgctgt ggtgtcagtt ccggatgcct
    61 catcgccagg ggcgcgccgc agccacccac cctccggacc gcggcagctg ctgacccgcc
  121 atcgccatgg cccgcgggaa agccaaggag gagggcagct ggaagaaatt catctggaac
  181 tcagagaaga aggagtttct gggcaggacc ggtggcagtt ggtttaagat ccttctattc
  241 tacgtaatat tttatggctg cctggctggc atcttcatcg gaaccatcca agtgatgctg
  301 ctcaccatca gtgaatttaa gcccacatat caggaccgag tggccccgcc aggattaaca
  361 cagattcctc agatccagaa gactgaaatt tcctttcgtc ctaatgatcc caagagctat
  421 gaggcatatg tactgaacat agttaggttc ctggaaaagt acaaagattc agcccagagg
  481 gatgacatga tttttgaaga ttgtggcgat gtgcccagtg aaccgaaaga acgaggagac
  541 tttaatcatg aacgaggaga gcgaaaggtc tgcagattca agcttgaatg gctgggaaat
  601 tgctctggat taaatgatga aacttatggc tacaaagagg gcaaaccgtg cattattata
  661 aagctcaacc gagttctagg cttcaaacct aagcctccca agaatgagtc cttggagact
  721 tacccagtga tgaagtataa cccaaatgtc cttcccgttc agtgcactgg caagcgagat
  781 gaagataagg ataaagttgg aaatgtggag tattttggac tgggcaactc ccctggtttt
  841 cctctgcagt attatccgta ctatggcaaa ctcctgcagc ccaaatacct gcagcccctg
  901 ctggccgtac agttcaccaa tcttaccatg gacactgaaa ttcgcataga gtgtaaggcg
  961 tacggtgaga acattgggta cagtgagaaa gaccgttttc agggacgttt tgatgtaaaa
1021 attgaagtta agagctgatc acaagcacaa atctttccca ctagccattt aataagttaa
1081 aaaaagatac aaaaacaaaa acctactagt cttgaacaaa ctgtcatacg tatgggacct
1141 acacttaatc tatatgcttt acactagctt tctgcattta ataggttaga atgtaaatta
1201 aagtgtagca atagcaacaa aatatttatt ctactgtaaa tgacaaaaga aaaagaaaaa
1261 ttgagccttg ggacgtgccc atttttactg taaattatga ttccgtaact gaccttgtag
1321 taagcagtgt ttctggcccc taagtattgc tgccttgtgt attttattta gtgtacagta
1381 ctacaggtgc atactctggt catttttcaa gccatgtttt attgtatctg ttttctactt
1441 tatgtgagca aggtttgctg tccaaggtgt aaatattcaa cgggaataaa actggcatgg
1501 taattttttt tttttgtttg ttttttgttt tttggctctt tcaaaggtaa tggcccatcg
1561 atgagcattt ttaacatact ccatagtctt ttcctgtggt gttaggtctt tatttttatt
1621 tttttcctgg gggctggggt gggggtttgt catgggggaa ctgcccttta aattttaagt
1681 gacactacag aaaaacacaa aaaggtgatg ggttgtgtta tgcttgtatt gaatgctgtc
1741 ttgacatctc ttgccttgtc ctccggtatg ttctaaagct gtgtctgaga tctggatctg
1801 cccatcactt tggcctaggg acagggctaa ttaatttgct ttatacattt tcttttactt
1861 tccttttttc ctttctggag gcatcacatg ctggtgctgt gtctttatga atgttttaac
1921 cattttcatg gtggaagaat tttatattta tgcagttgta caattttatt tttttctgca
1981 agaaaaagtg taatgtatga aataaaccaa agtcacttgt ttgaaaataa atctttattt
2041 tgaactttat aaaagcaatg cagtacccca tagactggtg ttaaatgttg tctacagtgc
2101 aaaatccatg ttctaacata tgtaataatt gccaggagta cagtgctctt gttgatcttg
2161 tattcagtca ggttaaaaca acggacaata aaagaatgaa ccgaattc
```

(SEQ ID NO: 8) GenBank Accession X03747 version 1, incorporated herein by reference. Exemplary regions or fragments of Na⁺/K⁺-ATPase include bases 127-1038, 598-600, 919-921, 1485-1490, 2001-2006, 2026-2031, and 2187-2193.

### Binding Ligands

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; preferably ≤100 nM and most preferably ≤10 nM.

Antibodies can be produced according to any method known in the art. Methods of preparing monoclonal antibodies are known in the art. For example, monoclonal antibodies may be prepared using hybridoma methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a full length protein or a fragment thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (see pp. 59-103 in Goding (1986) Monoclonal Antibodies: Principles and Practice Academic Press) Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some examples the antibodies to an epitope for an interested protein as described herein or a fragment thereof are humanized antibodies. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al. 1986. Nature 321:522-525; Riechmann et al. 1988. Nature 332:323-329; Presta. 1992. Curr. Op. Struct. Biol. 2:593-596). Humanization can be essentially performed following methods of Winter and co-workers (see, e.g., Jones et al. 1986. Nature 321:522-525; Riechmann et al. 1988. Nature 332:323-327; and Verhoeyen et al. 1988. Science 239:1534-1536), by substituting rodent CDRs or CDR sequences for the corresponding sequences of a human antibody. Accordingly, such humanized antibodies are chimeric antibodies (e.g., U.S. Pat. No. 4,816,567), wherein substantially less than an intact human variable domain has been substituted by the corresponding sequence from a non-human species.

In another example the antibodies to an epitope of an interested protein as described herein or a fragment thereof are human antibodies. Human antibodies can also be produced using various techniques known in the art, including phage display libraries (Hoogenboom and Winter. 1991. J. Mol. Biol. 227:381-388; Marks et al. 1991. J. Mol. Biol. 222:581-597) or the preparation of human monoclonal antibodies (e.g., Cole et al. 1985. Monoclonal Antibodies and Cancer Therapy Liss; Boerner et al. 1991. J. Immunol. 147(1):86-95). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in most respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al. 1992. Bio/Technology 10:779-783; Lonberg et al. 1994. Nature 368:856-859; Morrison. 1994. Nature 368:812-13; Fishwild et al. 1996. Nature Biotechnology 14:845-51; Neuberger. 1996. Nature Biotechnology 14:826; Lonberg and Huszar. 1995. Intern. Rev. Immunol. 13:65-93. U.S. Pat. No. 6,719,971 also provides guidance to methods of generating humanized antibodies.

Exemplary human AF-20 antibodies include, but are not limited to, antibodies commercially available. AF20 monoclonal antibody (mAb) was produced and characterized as previously described (Wilson B et al. Proc Natl Acad Sci 1988;85:3140-4 , Moradpour D et al. Hepatology 1995;22:1527-1537 , and Wands JR, et al. J Viral Hepat 1997;4 Suppl 2:60-74). The following antibodies were obtained commercially: anti-TFR1 (CD71) (Santa Cruz Biotechnology, Inc.; sc-32272), anti-HSP90 (EMD Millipore; 05-594), anti-N⁺/K⁺ ATPase (abcam, ab7671) and anti-DDK (Origene; TA100011).

### EXAMPLES

### Example 1

### Comparison of AF20 Protein Levels Among Cell Lines of Diverse Origins

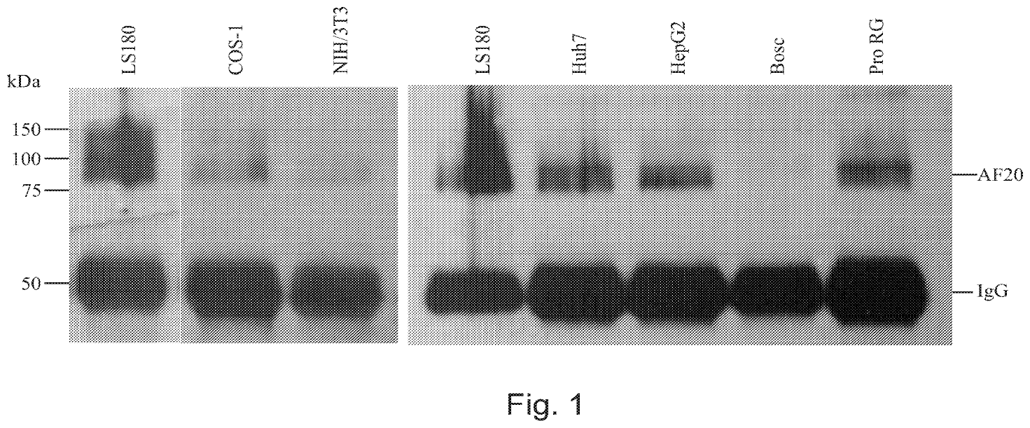

A robust AF20 expression in hepatoma and colon cancer cell lines was previously demonstrated (Wilson B, et al., Proc Natl Acad Sci 1988;85:3140-4 and Moradpour D et al., Hepatology 1995;22:1527-1537). IF staining indicated that AF20 antigen is localized on cell surface of hepatoma cells. Quantitative analysis revealed highest levels of AF20 antigen in hepatoma cell lines such as FOCUS and Huh7, as well as LS180, a colon cancer cell line. In contrast, NIH 3T3 and COS-1 cells showed no or little AF20 expression (Moradpour D et al., Hepatology 1995;22:1527-1537). To compare expression level of AF20 in cancer cells of diverse origins, quantitative IP-Western blot analysis was performed. AF20 expression was high in LS180, Huh7, HepG2, and proliferating HepaRG cells, a liver stem cell line, but low in Bosc, Cos-1 (human and monkey kidney), and NIH 3T3 (mouse embryonic fibroblast) cells (FIG. 1). These findings are highly consistent with the previous reports.

Plasmids, cell culture and expression methods: Expression constructs for TFR1 (Myc-DDK-tagged, RC200980, NM_003234.1), HSP90 (untagged, SC108085, NM_007355.2), and Na⁺/K⁺ ATPase (Myc-DDK-tagged, RC201009, NM_000701) were purchased from Origene. Human hepatoma cell line Huh7 (stock), human kidney cell line BOSC (ATCC), and monkey kidney cell line COS-1 (ATCC) were cultured in Dulbecco's Modified Eagle Medium (DMEM) supplemented with 10% fetal bovine serum (FBS). HepG2 (human hepatoma, ATCC), LS180 (human colon cancer, ATCC), and NIH 3T3 (mouse fibroblast, ATCC) cells were cultured in EMEM supplemented with 10% FBS and 1% non-essential amino acids. HepaRG (hepatic cell line, from Dr. Christian Trepo, Lyon, France) was cultured in William's E medium supplemented with 10% FBS, bovine insulin (5 μg/ml) and 7×10⁻⁵ M hydrocortisone. Cells grown in 6-well or 24-well plates with cover slips were transfected with 1 μg or 0.25 μg of TFR1 or other cDNA expression constructs using polyamine as a carrier (Mirus), and harvested 2 days later for Western blot analysis or fixed for immunofluorescent (IF) staining.

IP-Western blot analysis methods: Cell lysate was incubated at 4° C. overnight with AF20 antibody immobilized on protein G beads. After washing with PBS, bound proteins were separated in 8% or 10% SDS-PAGE, transferred to polyvinylidene fluoride (PVDF) membrane, blocked with 3% milk in PBS supplemented with 0.05% Tween 20 (PBST), and incubated with AF20 antibody overnight in the same solution. After washing with PBS, the membrane was incubated at room temperature (RT) for 1 hr with anti-mouse antibody conjugated with horseradish peroxidase (HRP). Signals were revealed by enhanced chemiluminescence (ECL) followed by exposure to X-ray film.

IF staining methods: Cells grown on cover slips were fixed at −20° C. with acid ethanol (5% acetic acid glacial, 95% ethanol) for at least 2 hrs. IF staining was performed using Immunostaining kit (Active Motif, 15251). Briefly, after washing with PBS and Maxwash, fixed cells were incubated successively, at 4° C. for lhr, with Maxblock, primary antibody in Maxbind, and secondary antibody in Maxbind. After washing with Maxwash, the cover slips were transferred onto glass slide using mounting solution containing DAPI, and signals were examined under UV microscope.

Immunohistochemistry methods: Formalin fixed, paraffin-embedded sections were deparaffinized by xylene followed by submerged in preheated Antigen Unmasking Solution H-3300 (Vector Laboratories, California) in a pressure cooker (Nordic Ware, Minnesota) for 2 minutes. Sections were cooled to room temperature in a water bath and then submerged in an endogenous peroxidase blocking solution containing 3% H₂O₂ in methanol for 30 minutes. The rest of the staining process, including blocking, primary and secondary antibody incubation, and peroxidase-labeling, was performed using the Elite ABC Kit PK-6102 (Vector Laboratories, California) according to manufacturer's instruction. Primary antibody was diluted 1:50-1:500 and incubated at 4° C. overnight. Sections were washed in PBST between each incubation step. Color development was done using DAB Peroxidase Substrate SK-4103 (Vector Laboratories, California). Developed sections were counterstained by hematoxylin. Finally, sections were dehydrated using a reversed ethanol gradient followed by xylene.

### Example 2

### AF20 mAb Immunoprecipitated TFR1 and Other Host Proteins

A combination of ion-exchange chromatography and affinity chromatography was used to purify AF20 protein from Huh7 cells. The cell lysate was loaded onto DEAE-cellulose column, and bound proteins were eluted sequentially with 100, 200, and 400 mM NaCl solution (FIG. 2A). Surprisingly, IP-Western blot analysis revealed presence of AF20 antigen in protein peaks from all the three NaCl concentrations (FIG. 2B). Protein peaks of the three NaCl concentrations were pooled for concentration by Centricon-30. After separation in SDS-PAGE, the large gel was stained with Coomassie blue. Three protein bands in the range of 90 to 110 kDd were visible (FIG. 2C). They were cut out and subject to mass spectrometry as detailed in Materials and Methods. The results, as shown in Table 1 (below), revealed presence of three proteins of similar molecular weights from Huh7 cells: transferrin receptor 1 (TFR1), heat shock protein 90 (HSP90), and Na⁺/K⁺ ATPase or Mg++ ATPase. Using each of the three bands for proteomic analysis still revealed presence of all the three proteins Similar results were obtained using cell lysate from FOCUS, LS180, and HepG2 cells, although ATPase was not always present (Table 1). Proteins from a single NaCl concentration were not used for proteomic analysis.

TABLE 1
Protein sequencing results

| Cell line | Protein identity | ID | MW |
|---|---|---|---|
| *FOCUS/LS180 | | | |
| 180 Kda: | TFR1 | gi37433 | 84848 |
| 90-100 Kda: | Na⁺/K⁺ ATPase | gi21361181 | 112842 |
| | HSP gp96 precursor | gi15010550 | 90138 |
| | TFR1 | gi37433 | 84848 |
| | HSP 90-β | gi20149594 | 83212 |
| | HSP 90-α2 | gi61656603 | 98052 |
| Huh7 | Mg⁺⁺ ATPase (Pseudomonas) | gi395497517 | 99809 |
| | HSP 90 | gi306891 | 83242 |
| | HSP 90-α | gi12082136 | 83046 |
| LS180 | TFR1 | P02786 | 84818 |
| | HSP 90-α | P07900 | 84607 |
| HepG2 | TFR1 | P02786 | 84818 |
| | HSP 90-β | P08238 | 83212 |
| | Na⁺/K⁺ ATPase subunit α1 | P05023 | 112824 |
| | HSP 90-α | P07900 | 84607 |
| FOCUS | TFR1 | P02786 | 84818 |
| | HSP 90-β | P08238 | 83212 |

*Mixed lysate from FOCUS and LS180 cells

Purification and identification methods: Two grams of cell pellet was re-suspended in water supplemented with a protease cocktail, followed by four cycles of freezing/thawing in dry ice/methanol bath and 37° C. water bath, respectively. Ten times concentrated phosphate buffered saline (PBS) was added to a final concentration of lx. After centrifugation at 14,000 rpm for 10 min, cell lysate was mixed with 1M Tris-HCL, pH 8.0 to a final concentration of 50 mM, and loaded onto a column containing 20 ml DEAE-cellulose. After flow-through, the column was washed three times with 20 ml each of 50 mM Tris pH 8.0. Proteins bound to DEAE cellulose column were eluted successively with 100 mM, 200 mM, and 400 mM NaCl prepared in 50 mM Tris, pH 8.0. Each fraction was collected and BCA assay was performed to measure protein concentration. Protein peaks were collected and subject to immunoprecipitation (IP) using AF20 antibody followed by Western blot with the same antibody. Fractions containing AF20 antigen were pooled for large scale IP followed by separation in 10% SDS-polyacrylamide gel (PAGE). Protein bands(s) corresponding to the size of AF20 (90-110 kDa) were excised and subject to trypsin digestion, followed by peptide separation and mass spectrometry (MS) analysis on a LTQ Orbitrap mass spectrometer at Keck Biotechnology Resource Laboratory, Yale University.

### Example 3

### Validation of TFR1 as an AF20 Antigen

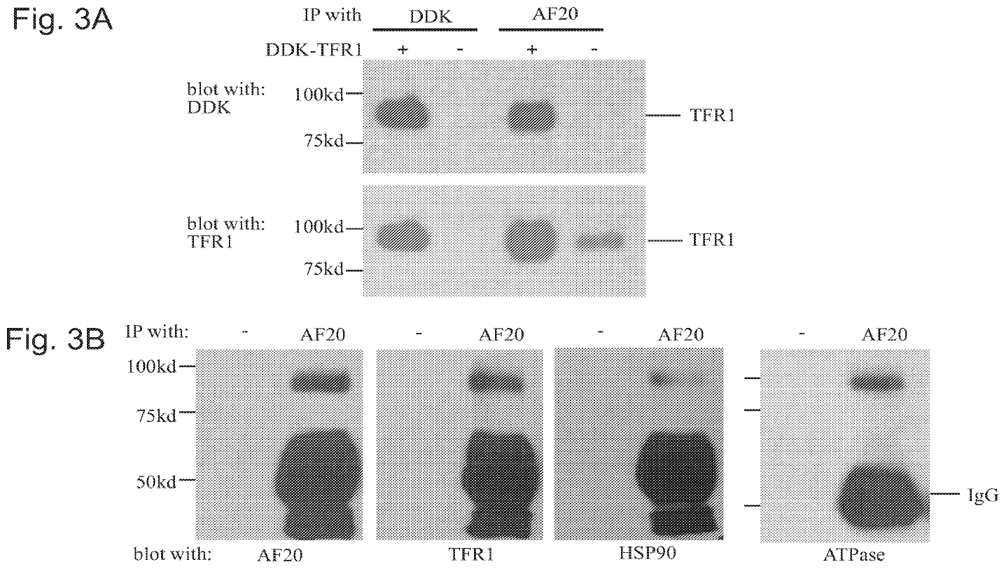

The AF20 antigen corresponded to a single host protein. The simultaneous purification of TFR1, HSP90, and ATPase by the AF20 mAb, as well as the presence of AF20 antigen in three concentrations of NaCl, was most likely a consequence of protein complex formation. To establish which one was the bona fide AF20 antigen, in vitro reconstitution experiments were performed using expression constructs for TFR1 (DDK tagged), Na⁺/K⁺ ATPase (DDK tagged) and HSP90 (untagged). DDK-tagged TFR1 expressed in Huh7 cells through transient transfection could be detected by IP—Western blot analysis using the DDK antibody as expected (FIG. 3A, upper left panel). Interestingly, substituting the DDK antibody with AF20 antibody for IP did not compromise detection of the exogenous TFR1 by the DDK antibody in Western blot (FIG. 3A upper right panel).

Reprobing the same blot with anti-TFR1 antibody revealed that endogenous TFR1 could be pulled down by the AF20 but not by the DDK antibody from non-transfected cells (FIG. 3A, lower panel). In the non-transfected Huh? cells, TFR1, HSP90, and ATPase could all be pulled down by the AF20 antibody, but not by the empty protein A/G beads (FIG. 3B).

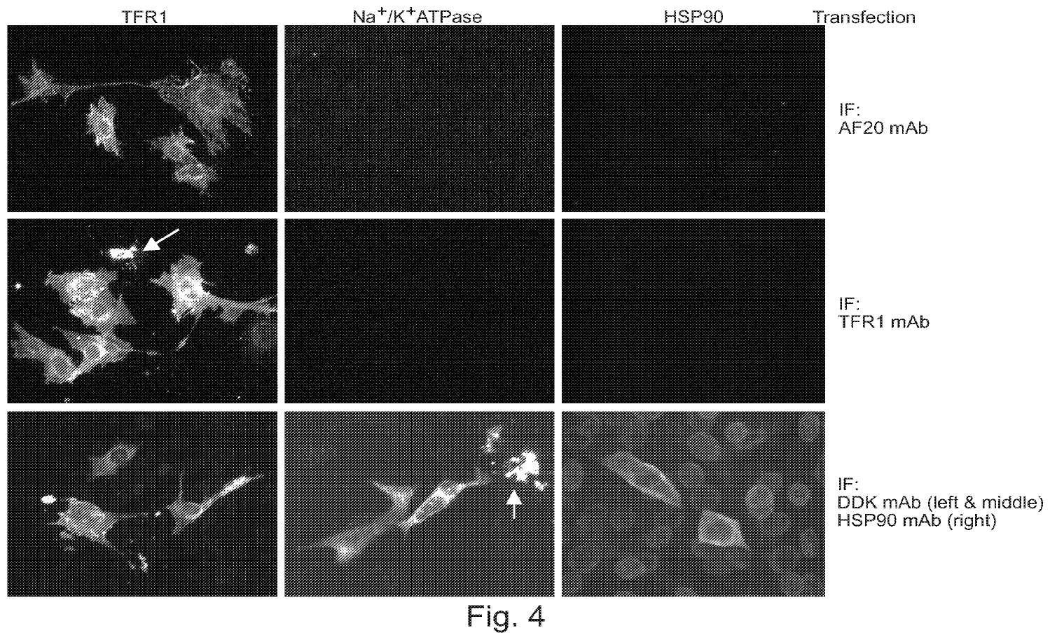

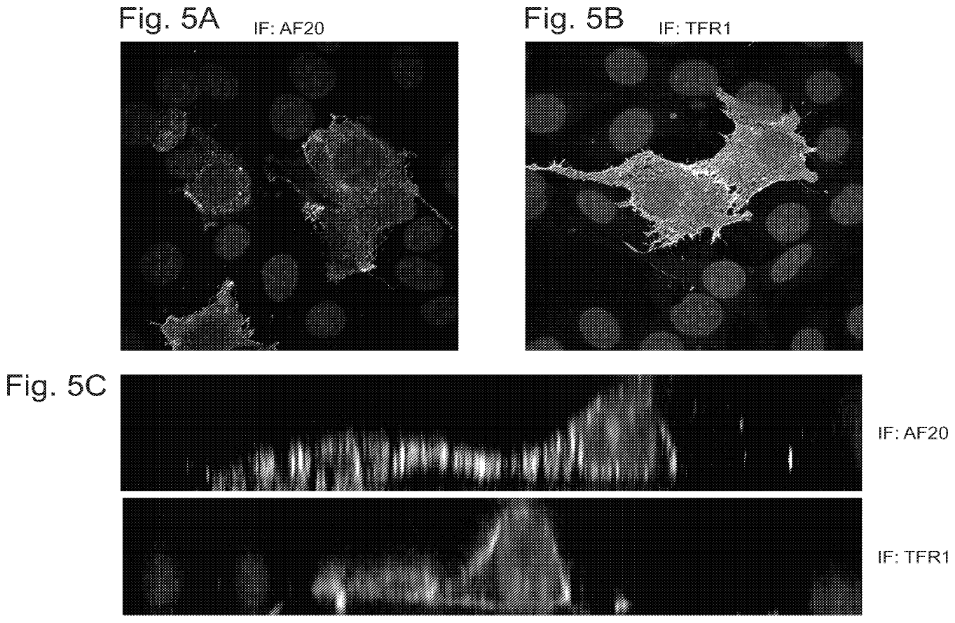

In a different approach, cDNAs were transfected encoding DDK tagged TFR1 and Na⁺/K⁺ ATPase, or untagged HSP90 into NIH 3T3 cells, a cell line with little endogenous AF20 expression (FIG. 1). Strong IF staining by AF20 antibody was documented in TFR1 but not Na⁺/K⁺ ATPase or HSP90 transfected NIH 3T3 cells (FIG. 4), despite the fact that these two proteins were readily stained by DDK mAb or HSP90 antibody (FIG. 4). Moreover, the AF20 antibody and TFR1 antibody revealed similar staining pattern in TFR1 transfected cells (FIG. 4 left and FIGS. 5A-5C).

These findings strongly implicated TFR1, rather than Na⁺/K⁺ ATPase or HSP90, as the AF20 antigen. Another interesting observation was that TFR1 transfected to NIH 3T3 cells exhibited not only cell surface localization, but also perinuclear staining as revealed by confocal microscopy (FIG. 4 and FIGS. 5A-5C). Moreover, from 2D or 3D view the staining by AF20 mAb and TFR1 mAb in TFR1 transfected cells was indistinguishable (FIGS. 5A-5C). Overexpression of the three proteins by transient transfection caused toxicity as indicated by morphological changes including cell shrinkage and elongation when compared with non-transfected cells (FIG. 4).

### Example 4

### Holo but not Apo Transferrin Could Disrupt AF20 Antibody Binding to TFR1

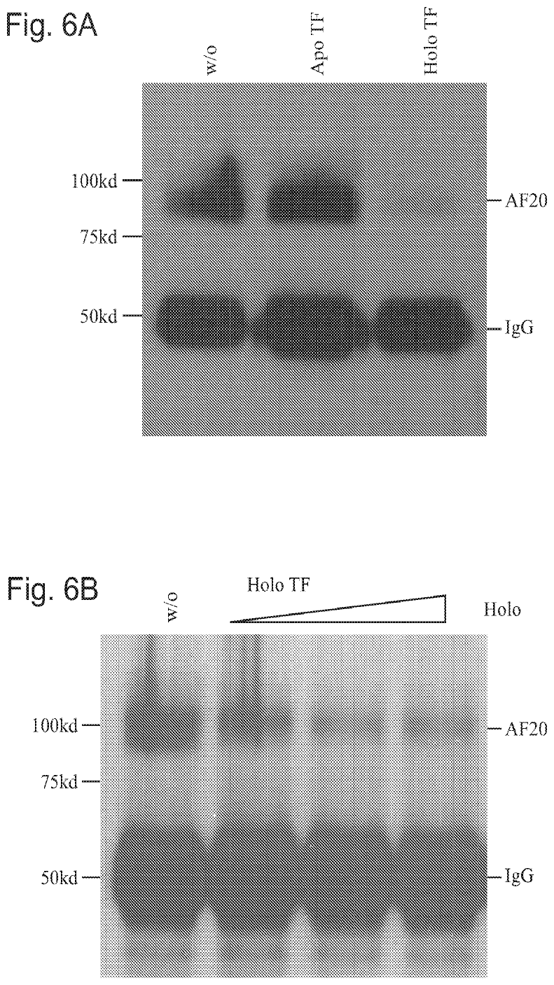

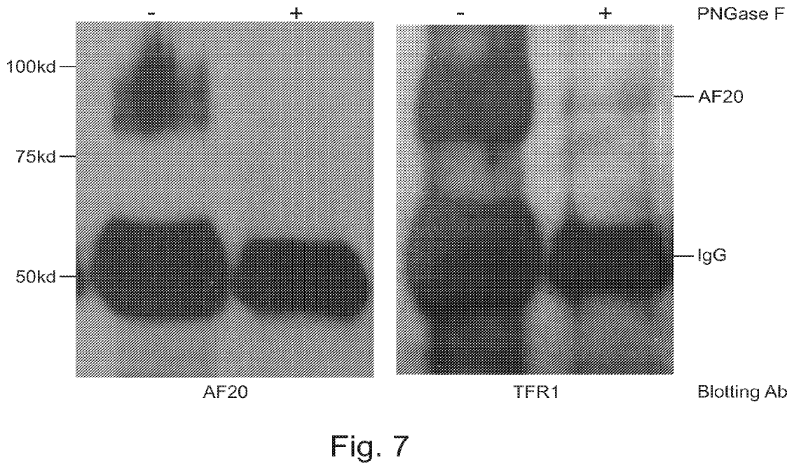

Diferric (holo) but not iron free (apo) transferrin is the ligand of TFR1 at neutral pH [5], two forms of transferrin were compared for their impact on AF20 antigen—antibody interaction. Cell lysate was immunoprecipitated with AF20 antibody in the presence or absence of transferrin, followed by Western blot with AF20 antibody. Holo but not apo transferrin inhibited TFR1 precipitation by the AF20 antibody (FIG. 6A). Increasing the concentration of holo transferrin from 3.3 to 66 μg/ml caused a dose-dependent inhibition of the antibody—antigen interaction (FIG. 6B). Further increase in the concentration of holo transferrin (≥66 μg/ml) during immunoprecipitation did not further reduce the antigen-antibody reaction.

### Example 5

### Deglycosylation of the AF20 Antigen Abolished its Affinity for AF20 Antibody

TFR1 contains three sites for N-linked glycosylation (Reckhow CL and Enns CA J Biol Chem 1988;263:7297-7301; hereby incorporated by reference). To determine whether N-linked glycosylation is essential for TFR1 recognition by the AF20 antibody, proteins pulled down by the AF20 antibody were treated with PNGase F. This enzyme cleaves between the innermost GLcNAc and asparagine residues of high mannose, hybrid, and complex oligosaccharides. Deglycosylated AF20 antigen could be no longer be detected by the AF20 antibody in the Western blot (FIG. 7, left panel), even after prolonged exposure. On the other hand, TFR1 antibody detected both glycosylated and deglycosylated forms of TFR1, although the signal intensity was much reduced for the deglycosylated form (FIG. 7, right panel). Alternatively, it is possible that deglycosylation makes TFR1 unstable with the intact protein dropped below the detection limit.

Protein deglycosylation methods: Huh7 cell lysate was subject to IP with AF20 antibody as described above. After washing with PBS, bound proteins were incubated with PNGase F at 37° C. for at least 1 hr under conditions recommended by the Manufacturer. Samples without addition of the enzyme were incubated in parallel for controls. Next, samples were loaded to 10% SDS-PAGE for Western blot analysis using AF20 and TFR1 mAbs, respectively. PNGase F was purchased from New England Biolabs (P0704S). Apo transferrin and holo transferrin were purchased from EMB Millipore (616395 and 616397).

### Example 6

### Evaluation of TFR1 as a Biomarker for Early Detection of Malignant Transformation

AF20 antigen has been detected in majority of hepatoma and colon cancer cell lines (Wilson B, et al., Proc Natl Acad Sci 1988;85:3140-4). To correlate AF20 expression with the stage of cancer development, immunostaining was performed in four paired samples including normal colon tissue, colon polyps, and colon cancer. While AF20 was undetectable in normal colon tissues, it was clearly detectable in polyps and strongly detected in colon cancer from all four pairs of samples (FIG. 8 for two representative pairs). In addition, relationship between AF20 and TFR1 was sought by their localization in colon cancer tissues. Three pairs of consecutive tissue sections from normal colon and colon cancer were stained with AF20 and TFR1 mAb, respectively. Both AF20 and TFR1 antibodies revealed strong signals in colon cancer with similar localization (FIG. 9B). In contrast, no signal was detected from normal colon tissues by either antibody (FIG. 9A). Together with immunostaining of TFR1 cDNA transfected cells by AF20 mAb (FIG. 4), this finding strongly implicates TFR1 as the AF20 antigen.

### Example 7

### Therapeutic Advantages

Therapeutic advantages include a monoclonal antibody that identifies malignant cells. The antibody can be used for clinical diagnosis, delivery of an anti-tumor agent and imaging of a tumor in the body. Furthermore, the invention can aid in the diagnosis and treatment of cancer—it is useful for physicians and other healthcare personnel that treat cancer patients.

The compounds and methods described have broad implications for human cancer diagnosis and therapy and are used as an imaging agent, as a carrier of a drug or isotope, (e.g., U.S. Pat. No. 5,703,213, col. 21-22, herein incorporated by reference) since once the antibody binds to the antigen complex, is internalized into the cell and are used as a diagnostic marker of cell transformation such as screening for dysplastic cellular changes in long standing ulcerative colitis which has a high risk of developing colon cancer. It might also be used to evaluate prognosis with respect to early tumor reoccurrence and overall survival.

Identification of the extensively characterized AF20 antigen as glycosylated TFR1 explains features of AF20, including its homodimeric structure, cell surface localization, rapid endocytosis of antigen-antibody complex, overexpression in liver and colon cancers, and potential for cancer therapy. It also led to novel findings such as blocking of AF20 antigen-antibody interaction by diferric transferrin. In addition, the current study also revealed ability of TFR1 to form a complex with HSP90 and/or Na⁺/K⁺ ATPase or Mg++ ATPase. In this regard, the presence of AF20 antigen (TFR1) at eluents from 100 mM, 200 mM, and 400 mM NaCl (FIG. 2A) might reflect its different molecular forms (free form, HSP90 bound, ATPase bound, or tertiary complex). As shown in FIG. 5C, overexpression of TFR1 alone led to its perinuclear staining in addition to cell surface localization. The binding site for AF-20 is a post translation modification of the TFR1 protein. Antibodies that bind to a TFR1 antigen with an aberrant post translational modification, e.g., glycosylation (compared to the normal, wild type TFR1) are useful for cancer diagnosis and monitoring response to anti-tumor therapy.

### Other Embodiments

While the invention has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank, NCBI, UniProt, or other submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the disclosure encompassed by the appended claims.

What is claimed:

1. A composition or method comprising a purified human transferrin receptor 1 (TFR1) protein or a purified cell surface glycosylated peptide fragment thereof, for use as a biomarker for detection of malignancy.

2. The composition of claim 1, wherein said TFR1 protein or peptide fragment thereof comprises glycosylation at an aberrant site compared to a wild type TFR1 protein.

3. The compositon of claim 1, wherein said protein or peptide fragment does not comprise a N-linked glycosylation at amino acid position 251, 317, or 727 of SEQ ID NO:1.

4. The composition of claim 1, wherein said protein or peptide fragment comprises an N-linked glycosylation at amino acid position 50 or 55 of SEQ ID NO:1.

5. The composition of claim 1, wherein said protein or peptide fragment comprises an O-linked glycosylation at amino acid position 104 of SEQ ID NO:1.

6. A purified monoclonal antibody that binds to a human transferrin receptor 1 (TFR1) for use in immunotherapy, as a vehicle for delivery of anti-tumor compounds, or as a diagnostic agent, wherein said antibody binds to an aberrant glycosylation site of said TFR1.

7. The monoclonal antibody of claim 6, wherein said TFR1 comprises glycosylation at an aberrant site compared to a wild type TFR1 protein.

8. The monoclonal antibody of claim 6, wherein said antibody comprises AF-20.

9. The monoclonal antibody of claim 6, wherein said antibody does not comprise AF-20.

10. The composition or method of claim 1, wherein said TFR1 or glycosylated peptide thereof, is in the absence of other compounds with which it naturally occurs in a mammal, said compounds being selected from the group consisting of Heat Shock Protein 90 (HSP90) and/or a Na⁺/K⁺ ATPase or Mg++ ATPase (Transporting ATPase).

11. A method for delivering a cargo molecule to a subject, the method comprising:
administering an antibody conjugated to said cargo molecule to the subject,
wherein the antibody binds to a glycosylated human transferrin receptor-1 antigen, and
wherein said cargo molecule is selected from the group consisting of a therapeutic agent or a detectable label.

12. The method of claim 11, wherein said therapeutic agent comprises a cytotoxic compound.

13. The method of claim 11, wherein said detectable label comprises a fluorescent compound or a radioisotope.

14. The method of claim 11, wherein said subject is characterized as comprising a malignancy.

15. The method of claim 11, wherein said antibody comprises AF-20.

16. The method of claim 11, wherein said antibody does not comprise AF-20.

17. The composition or method of claim 1, wherein said receptor or peptide thereof comprises asparagine (N)-linked glycosylation.

18. A purified glycosylated TFR1 antigen epitope comprising N-linked glycosylation at amino acid position 50 of SEQ ID NO:1 or amino acid position 55 of SEQ ID NO:1.

19. A purified glycosylated TFR1 antigen epitope comprising O-linked glycosylation at amino acid position 104 of SEQ ID NO:1.

20. The antigen epitope of claim 18 or 19, wherein said epitope comprises a length of at least 5 consecutive amino acids of SEQ ID NO:1.

21. A method of diagnosing a malignancy, comprising contacting a bodily tissue or fluid of a subject with an antibody that binds to TFR1, wherein said TFR1 comprises glycosylation at an aberrant site compared to a wild type TFR1 protein.

22. The method of claim 21, further comprising administering to said subject a cancer therapeutic composition.

23. The method of claim 11 or 21, wherein said subject comprises or is at risk of developing a colon cancer, a liver cancer, or a lung cancer.
